(19) United States
(12) Patent Application Publication
(10) Pub. No.: US 2025/0114581 A1
(43) Pub. Date: Apr. 10, 2025

(54) Dermal Patch for Delivering a Pharmaceutical

(71) Applicant: Satio, Inc., Boston, MA (US)

(72) Inventors: Namal NAWANA, Weston, MA (US); Ziad Tarik Al-Shamsie, San Diego, CA (US)

(73) Assignee: Satio, Inc., Boston, MA (US)

(21) Appl. No.: 18/985,616

(22) Filed: Dec. 18, 2024

Related U.S. Application Data
(62) Division of application No. 18/090,107, filed on Dec. 28, 2022, now Pat. No. 12,178,979.
Application No. 18/090,107 is a continuation-in-part of application No. 17/994,454, filed on Nov. 28, 2022, now Pat. No. 11,877,848.
Application No. 17/994,454 is a continuation of application No. 17/521,466, filed on Nov. 8, 2021, now Pat. No. 11,510,602.
Application No. 18/090,107 is a continuation-in-part of application No. 17/991,284, filed on Nov. 21, 2022, now Pat. No. 12,048,543.
Application No. 17/991,284 is a continuation-in-part of application No. 17/971,142, filed on Oct. 21, 2022, now Pat. No. 12,053,284.
Application No. 17/971,142 is a continuation-in-part of application No. 17/521,466, filed on Nov. 8, 2021, now Pat. No. 11,510,602.
Application No. 18/090,107 is a continuation-in-part of application No. 17/903,802, filed on Sep. 6, 2022.
Application No. 17/903,802 is a continuation of application No. 17/412,213, filed on Aug. 25, 2021, now Pat. No. 11,452,474.
Application No. 18/090,107 is a continuation-in-part of application No. 17/500,873, filed on Oct. 13, 2021, now Pat. No. 11,964,121.
(60) Provisional application No. 63/190,700, filed on May 19, 2021.
(60) Provisional application No. 63/174,956, filed on Apr. 14, 2021.

(51) Int. Cl. A61K 9/00; A61K 39/00

(52) U.S. Cl. CPC A61M 37/0015; A61M 5/14212; A61M 2037/0023; A61M 2037/0061; A61M 2202/30; A61M 2205/6063

(57) ABSTRACT

A dermal patch system for administering a pharmaceutical includes a vial that stores a pharmaceutical and a cartridge coupled to the vial. The cartridge includes a pull mechanism, a pump, a plurality of microneedles in communication with the vial. The microneedles are configured to move between an undeployed position to a deployed position. When pulled, the pull mechanism is configured to cause the microneedles to move from the undeployed position to the deployed position and cause the pump to pump the pharmaceutical from the vial and to the microneedles.

### RELATED APPLICATIONS

The present application is a divisional of U.S. patent application Ser. No. 18/090,107 filed on Dec. 28, 2022, which is a continuation-in-part of: (1) U.S. patent application Ser. No. 17/903,802 filed on Sep. 6, 2022, which is a continuation of U.S. application Ser. No. 17/412,213 filed on Aug. 25, 2021 (issued as U.S. Pat. No. 11,452,474), which claims the benefit of U.S. Provisional Application Nos. 63/190,700 and 63/174,956, filed on May 19, 2021 and Apr. 14, 2021, respectively; (2) U.S. patent application Ser. No. 17/500,873 filed on Oct. 13, 2021 (issued as U.S. Pat. No. 11,964,121); (3) U.S. patent application Ser. No. 17/994,454 filed on Nov. 28, 2022 (issued as U.S. Pat. No. 11,877,848), which is a continuation of U.S. patent application Ser. No. 17/521,466 filed on Nov. 8, 2021 (issued as U.S. Pat. No. 11,510,602); and (4) U.S. patent application Ser. No. 17/991,284 filed on Nov. 21, 2022 (issued as U.S. Pat. No. 12,048,543), which is a continuation-in-part of U.S. patent application Ser. No. 17/971,142 filed on Oct. 21, 2022 (now U.S. Pat. No. 12,053,284), which is a continuation-in-part of U.S. application Ser. No. 17/521,466 filed on Nov. 8, 2021 (issued as U.S. Pat. No. 11,510,602). Each of these applications is herein incorporated by reference in its entirety.

### TECHNICAL FIELD

The present teachings are generally directed to dermal patch systems (herein also referred to as dermal patches) that can be employed to deliver a pharmaceutical to a subject.

### BACKGROUND

Typically, administration of pharmaceuticals (e.g., vaccines) is carried out via a standard vial and a syringe. In large majority, the delivery is intramuscular. This mode of administration requires the expertise of a medical professional, which can limit the availability of life-saving pharmaceuticals to certain patient populations. For example, in many developing areas of the world where access to medical professionals may be limited, such conventional modes of parenteral administration may deprive large segments of the population from access to needed pharmaceuticals.

### SUMMARY

Aspects of the present disclosure address the above-referenced problems and/or others.

In one aspect, a dermal patch system for administering a pharmaceutical includes a vial that stores a pharmaceutical, and a cartridge coupled to the vial. The dermal patch includes a pull mechanism, a pump, and a plurality of microneedles in communication with the vial. In some embodiments, after a user properly positions the dermal patch system on the skin of a subject, a user can pull the pull mechanism which causes the dermal patch system to prime the microneedles with the pharmaceutical stored in the vial and move the microneedles from an undeployed position to a deployed position and causes the pump to pump the pharmaceutical from the vial. That is, the microneedles are configured to move between an undeployed position to a deployed position, and when pulled, the pull mechanism is configured to cause the pump to pump the pharmaceutical from the vial and to the microneedles and to cause the microneedles to move to the deployed position. In some embodiments, the pump is configured to prime the microneedles with an amount of pharmaceutical before the microneedles are moved to the deployed position for puncturing the subject's skin. In certain embodiments, the cartridge further includes a tube configured to carry the pharmaceutical from the vial to the plurality of microneedles.

In some embodiments, the pump can be a positive rotatable positive displacement pump and pulling the pull mechanism can cause the pump to rotate which causes the pump to force the pharmaceutical through the tube via positive displacement. In certain embodiments, the tube is a first tube, and the cartridge includes a second tube connected to the vial, and wherein rotation of the pump further causes the pump to force air into the vial through the second tube via positive displacement. In some embodiments, the cartridge also includes a latch that allows the pump to rotate in a first direction and prevents the pump from rotating in an opposite second direction. In certain embodiments, the pull mechanism is configured to move when then the cartridge has a first orientation and is prevented from moving when the cartridge has a different second orientation. In some embodiments the cartridge further includes a trigger that is configured to move from a first position to a second position when the pull mechanism is pulled. In the first position the trigger retains the microneedles in the undeployed position and in the second position, the trigger is configured to release the microneedles to a deployed position.

In certain embodiments, the pump is configured to move the trigger from the first position to the second position when the pull mechanism is pulled. In some embodiments the cartridge further includes an injection spring that is in a compressed state when the trigger is in the first position and is in an extended state when the trigger is in the second position. The injection spring is configured to move the microneedles to the deployed position when in the extended position. In certain embodiments, the cartridge further includes a retraction button configured to move the microneedles from the deployed position to the undeployed position. In some embodiments, the cartridge further includes a latch that prevents a user from removing the vial from the cartridge. In some embodiments, the pharmaceutical is a vaccine (e.g., Monkeypox vaccine, Flu vaccine, COVID-19 vaccine, Yellow Fever vaccine, Malaria vaccine, Dengue vaccine, etc.). In certain embodiments, the vial includes a fractional dose of the vaccine. In certain embodiments, the cartridge includes a quick response code that is associated with an electronic medical record.

In a related aspect a method for administering a pharmaceutical to a subject includes inserting a vial containing a pharmaceutical into a cartridge and attaching the cartridge to the subject's skin. The cartridge includes a pump and a plurality of microneedles. The method further includes pulling the pull mechanism to cause the microneedles to move from an undeployed position to a deployed position such that the microneedles puncture the skin of the subject when in the deployed position. Furthermore, pulling the pull mechanism causes the pump to pump the pharmaceutical to the microneedles thereby delivering the pharmaceutical to the subject. In some embodiments, the pharmaceutical is a vaccine. In certain embodiments, a fractional dose of the vaccine is delivered to the subject. In some embodiments, the method includes priming the microneedles by delivering a portion of the pharmaceutical to the microneedles before moving the microneedles to a deployed position. In certain embodiments, the cartridge further includes a tube that carries the pharmaceutical from the vial to the microneedles. In some embodiments, the pump pumps the pharmaceutical from the vial to the microneedles via positive displacement.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIGS. 1A, 1B, 2A, and 2B depict a dermal patch system in accordance with an exemplary embodiment;

FIGS. 3A, 3B, 4A, and 4B depict the dermal patch system without a cover in accordance with an exemplary embodiment;

FIGS. 5A, 5B, 6A, and 6B depict a cartridge of the dermal patch system in accordance with an exemplary embodiment;

FIGS. 7A and 7B-13A and 13B depict a cover of the dermal patch system in accordance with an exemplary embodiment;

FIGS. 14A and 14B-23 depict a base of the dermal patch system in accordance with an exemplary embodiment;

FIGS. 24A and 24B-26 depict a vial guide of the base of the dermal patch system in accordance with an exemplary embodiment;

FIGS. 27A and 27B-29A and 29B depict a handle retention feature of the base of the dermal patch system in accordance with an exemplary embodiment;

FIGS. 30A and 30B-33A and 33B depict a microneedle array housing of the dermal patch system in accordance with an exemplary embodiment;

FIGS. 34A and 34B-36 depict the cartridge of the dermal patch system without the cover and without the pump assembly in accordance with an exemplary embodiment;

FIGS. 37A and 37B depict a pull mechanism of the cartridge of the dermal patch system in accordance with an exemplary embodiment;

FIGS. 38A, 38B, 39A, and 39B depict a handle of the pull mechanism in accordance with an exemplary embodiment;

FIGS. 40A, 40B, 41A, and 41B depict a pump assembly of the cartridge of the dermal patch system in accordance with an exemplary embodiment;

FIGS. 42A and 42B-45A and 45B depict a trigger portion of the pump assembly of the cartridge of the dermal patch system in accordance with an exemplary embodiment;

FIGS. 46A and 46B-50A and 50B depict a pump portion of the pump assembly of the cartridge of the dermal patch system in accordance with an exemplary embodiment;

FIGS. 51A, 51B, 52A, and 52B depict a microneedle array assembly of the cartridge of the dermal patch system in accordance with an exemplary embodiment;

FIGS. 53A and 53B-56A and 56B depict a microneedle array holder of the microneedle array assembly in accordance with an exemplary embodiment;

FIG. 57 depicts an adhesive film of the microneedle array holder in accordance with an exemplary embodiment;

FIGS. 58A and 58B-61A and 61B depict a microneedle array of the microneedle array assembly in accordance with an exemplary embodiment;

FIGS. 62A and 62B depict the microneedle array holder with the adhesive film in accordance with an exemplary embodiment;

FIGS. 63A and 63B-67A and 67B depict a retraction button of the cartridge of the dermal patch system in accordance with an exemplary embodiment;

FIGS. 68A and 68B-70A and 70B depict a trigger of the cartridge of the dermal patch system in accordance with an exemplary embodiment;

FIG. 71 depicts a retraction button of the cartridge contacting a microneedle array assembly of the cartridge in accordance with an exemplary embodiment;

FIG. 72 depicts a ball of a pump assembly of the cartridge in a first position within the pump assembly in accordance with an exemplary embodiment;

FIG. 73 depicts a ball of a pump assembly of the cartridge in a second position within the pump assembly in accordance with an exemplary embodiment;

FIG. 74 depicts a trigger and a trigger portion of the pump assembly of the cartridge in a first position in accordance with an exemplary embodiment;

FIG. 75 depicts a trigger and a trigger portion of the pump assembly of the cartridge in a second position in accordance with an exemplary embodiment;

FIG. 76 depicts a microneedle array of the cartridge in an undeployed position in accordance with an exemplary embodiment;

FIG. 77 depicts a microneedle array of the cartridge in a deployed position in accordance with an exemplary embodiment;

FIG. 78 depicts a microneedle array of the cartridge in a deployed position in accordance with an exemplary embodiment;

FIG. 79 depicts the microneedle array of the cartridge in a retracted position in accordance with an exemplary embodiment;

FIG. 80 depicts the cartridge of the dermal patch system with a piezoelectric pump in accordance with an exemplary embodiment;

FIGS. 81 depicts a dermal patch system with a quick response (“QR”) code and a computer system that is in communication with an electronic medical record (“EMR”) database in accordance with an exemplary embodiment;

FIG. 82 schematically depicts a computer system in accordance with an exemplary embodiment;

FIG. 83 schematically depicts a cloud computer environment in accordance with an exemplary embodiment; and

FIG. 84 is a flow chart of a method for administering a pharmaceutical to a subject in accordance with an exemplary embodiment.

### DETAILED DESCRIPTION

The present disclosure generally relates to a dermal patch that may be utilized to deliver a pharmaceutical to a subject.

In some embodiments, a dermal patch may be used to deliver a pharmaceutical via a vial that can be attached to the dermal patch. Dermal patches disclosed herein may allow for the delivery of a pharmaceutical in a variety of environments (e.g., in the home, in the field, in a medical facility, etc.).

The term “about,” as used herein, denotes a deviation of at most 10% relative to a numerical value. For example, about 100 μm means in the range of 90 μm-110 μm.

The term “substantially,” as used herein, refers to a deviation, if any, of at most 10% from a complete state and/or condition.

The term “subject” as used herein refers to a human subject or an animal subject (i.e., chicken, pig, cattle, dog, cat, etc.).

The term “pharmaceutical” as used herein refers to a substance that is used in diagnosis, treatment, or prevention of a disease or a substance that restores, corrects, or modifies a biological function.

The term “fractional dose” as used herein refers to a dose of a pharmaceutical, particularly a vaccine, that is a fraction of a standard dose of that pharmaceutical (e.g., one-fifth, two-thirds, three-fourths, etc.) administered by a same or an alternative route (e.g., intradermally rather than subcutaneously or intramuscular, etc.). For example, if a standard dose of a vaccine includes a 100 μg dose, a fractional dose of that vaccine may include a 20 μg dose, a 25 μg dose, a 50 μg dose, a 75 μg dose, etc. With respect to a fractional dose of a vaccine, fractional dose vaccination may reduce a number of infections within a population while administering only a fraction of the dose of the vaccine to a number of subjects which in turn may allow more individuals to become vaccinated.

The term “transparent,” as used herein, indicates that light can substantially pass through an object (e.g., a window) to allow visualization of a material disposed behind the object. For example, in some embodiments, a transparent object allows the passage of at least 70%, or at least 80%, or at least 90% of visible light therethrough.

The term “needle” as used herein, refers to a component with a pointed tip that is configured to pierce an outer surface of an element (e.g., skin of a subject) to provide a passageway through the skin. A needle can be hollow to allow a fluid (e.gb., a pharmaceutical) to pass therethrough.

The term “microneedle” as used herein, refers to micron scaled needles used to administer a pharmaceutical. A microneedle can have length between about 1 mm and about 3 mm. Furthermore, the microneedles can administer a pharmaceutical to a subject at a rate of about 250 μl/min per microneedle. A microneedle can have a diameter between about 50 and about 350 microns wide or can have a gauge between 28 and 36 and can have a tip thickness between about 1 and about 25 microns.

The term “tube” as used herein, refers to hollow cylinder that provides a fluidic channel for transporting liquids or gases.

The present disclosure generally relates to a device, which is herein also referred to as a dermal patch or a dermal patch system, for delivering a pharmaceutical to a subject. In some embodiments discussed below, such a dermal patch system can include a cartridge that can be affixed to a subject's skin (e.g., via an adhesive layer), a separate vial containing a pharmaceutical can be attached to the cartridge and an at least one microneedles and more typically an array of microneedles disposed within the cartridge can be deployed to puncture the subject's skin and deliver the pharmaceutical to the subject. In some embodiments, the microneedles deliver the pharmaceutical intradermally. In some such embodiments, the dermal patch system is configured to deliver a single fractional dose of the pharmaceutical stored within the vial. As discussed in more detail below, the cartridge includes a pull mechanism that deploys the microneedle array and causes the cartridge to pump a dose of the pharmaceutical into the subject via the deployed microneedles. The cartridge can also include a push button mechanism. After a user administers the pharmaceutical, the user can push the push button mechanism into the cartridge which causes the microneedle array to retract into the cartridge which in turn allows the user to remove the cartridge from the subject. In this manner, the dermal patch system remains safe before it is engaged and after delivery of the pharmaceutical as in both cases the microneedle array is retained within the cartridge.

In some embodiments, the pull mechanism cannot be pulled unless the dermal patch system has a proper orientation on a subject's skin (e.g., a substantially vertical position). As such, the dermal patch system may administer the pharmaceutical only when the dermal patch has a given orientation. Providing a dermal patch system that administers a pharmaceutical when in a specific orientation may ensure that the subject receives all or substantially all of the pharmaceutical from a vial as the dermal patch may be aided by gravity when administering the pharmaceutical.

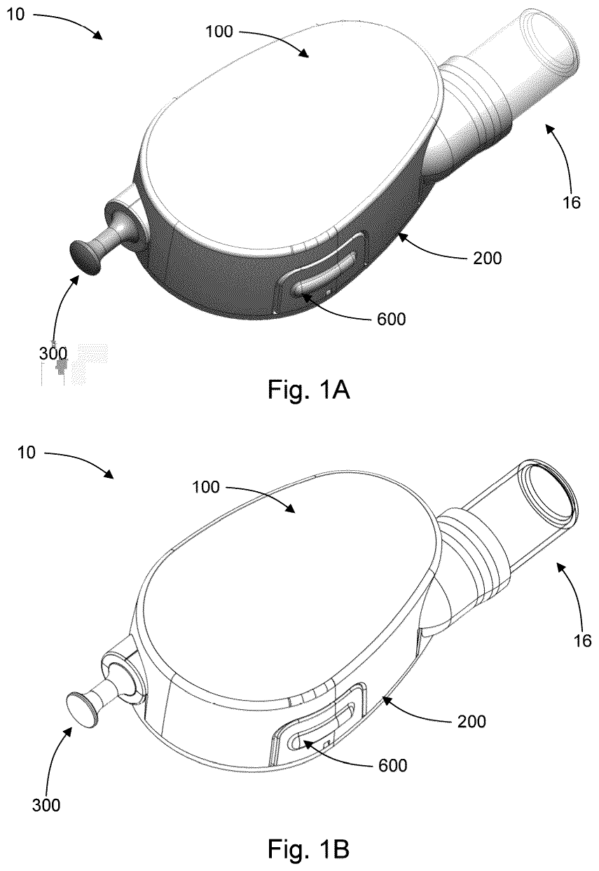

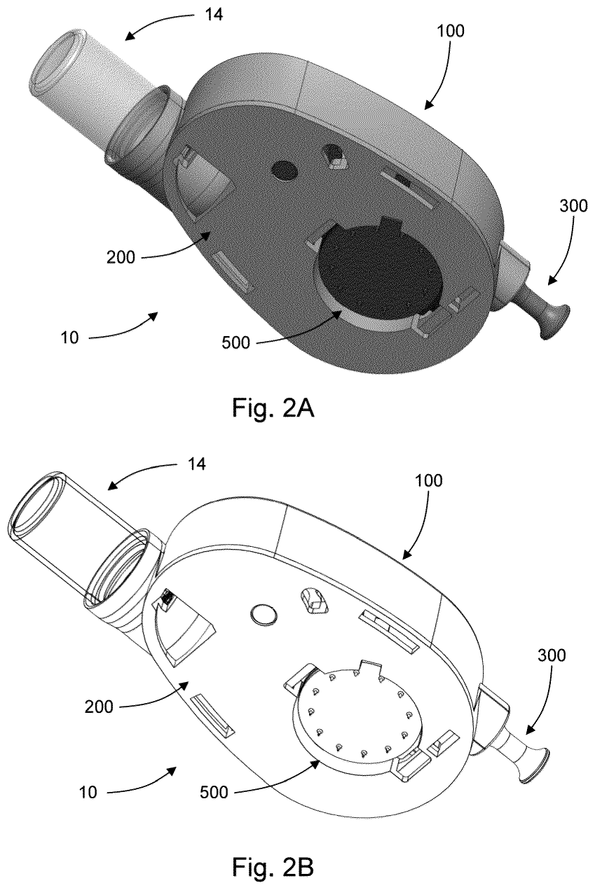

Referring now to FIGS. 1A, 1B-4A and 4B, a dermal patch system 10 is shown in accordance with an exemplary embodiment. In this embodiment, the dermal patch system 10 includes a cartridge 12 that can be affixed to a subject's skin via an adhesive layer. The dermal patch system 10 also includes a vial 14 that contains a pharmaceutical. The vial 14 can be inserted into the cartridge 12 for delivery of the pharmaceutical stored therein. In some embodiments, the vial 14 includes a single fractional dose of a pharmaceutical. More particularly, in some embodiments, the vial 14 includes a single fractional dose of a vaccine.

The cartridge 12 includes a cover 100 and a base 200 that can couple to the cover 100. For example, the cover 100 and the base 200 can be formed as two separate components that are removably coupled to one another (e.g., via a snap fitting). In other embodiments, the cover 100 and the base 200 form an integral unitary cartridge 12. In some of these embodiments, the cover 100 can be coupled to the base 200 via an adhesive, laser welding, etc.

The cartridge 12 may be formed using a variety of suitable materials including, but not limited to, polymeric materials (e.g., polyolefins, polyethylene terephthalate (PET), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, photocross linkable polymers, etc.). In some embodiments, some of the cover 100 may be formed of poly(dimethylsiloxane) (PDMS) to allow visibility of components disposed within the cartridge 12.

The cartridge 12 also includes a pull mechanism 300, a pump assembly 400 coupled to the pull mechanism 300, a microneedle array assembly 500, a retraction button 600, and a trigger 700. As will be discussed in further detail herein, when pulled, the pull mechanism 300 causes the trigger 700 to release the microneedle array assembly 500 thereby allowing microneedle array assembly 500 to move to a deployed position to puncture the skin of a subject. Furthermore, when pulled and after causing the microneedle array assembly 500 to deploy, the pull mechanism 300 causes the pump assembly 400 to pump the pharmaceutical from the vial 14 (when the vial 14 is coupled to the cartridge 12) through the microneedle array assembly 500into the subject. After the pharmaceutical has been administered to the subject, a user can push the retraction button 600, which causes the microneedle array assembly 500 to retract into the cartridge 12 which allows the dermal patch system 10 to be removed from the subject. Providing dermal patch system 10 which places the microneedle array assembly 500 in a stored position before and after use keeps the dermal patch system 10 in a safe state. In other words, the microneedle array assembly 500 is securely retained within the cartridge 12 when the cartridge is not in use.

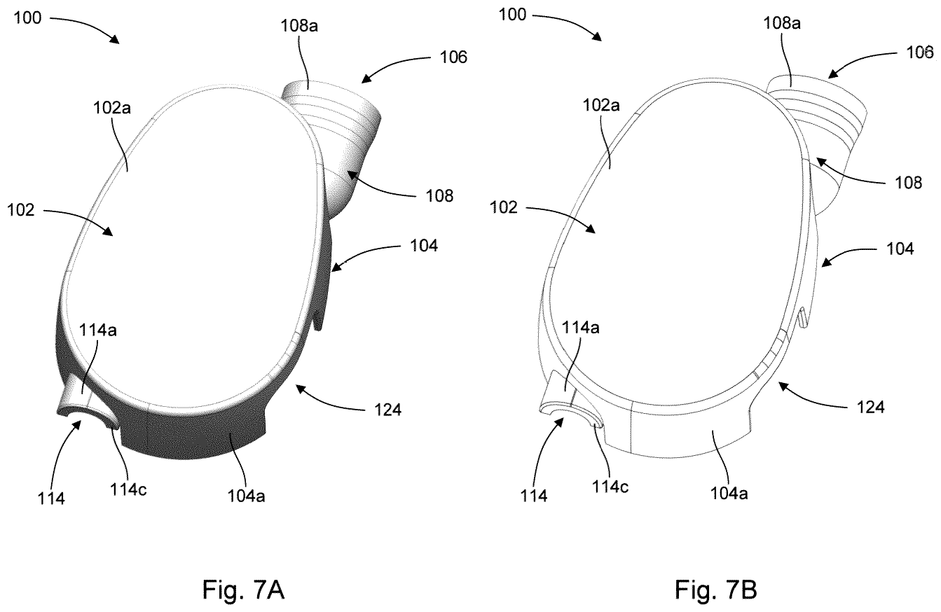

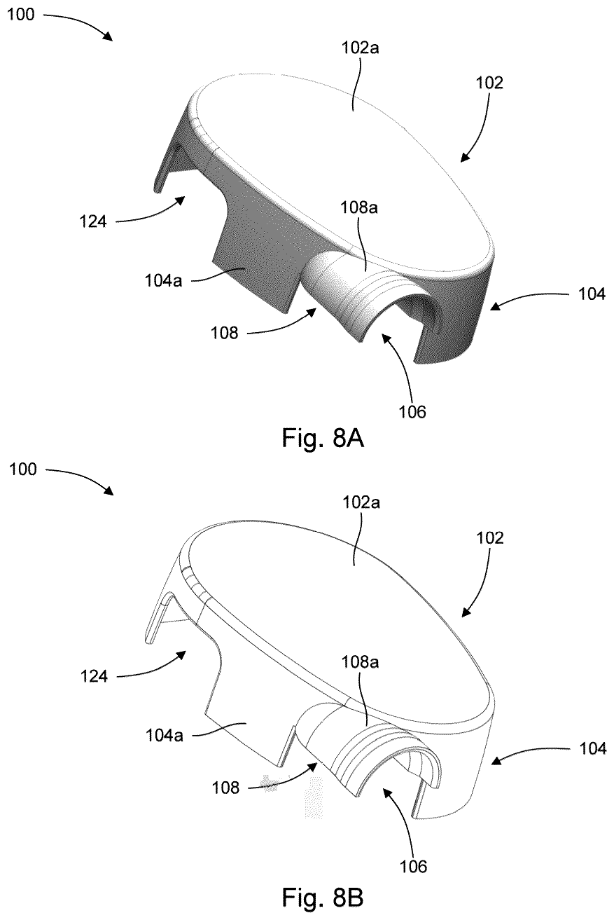

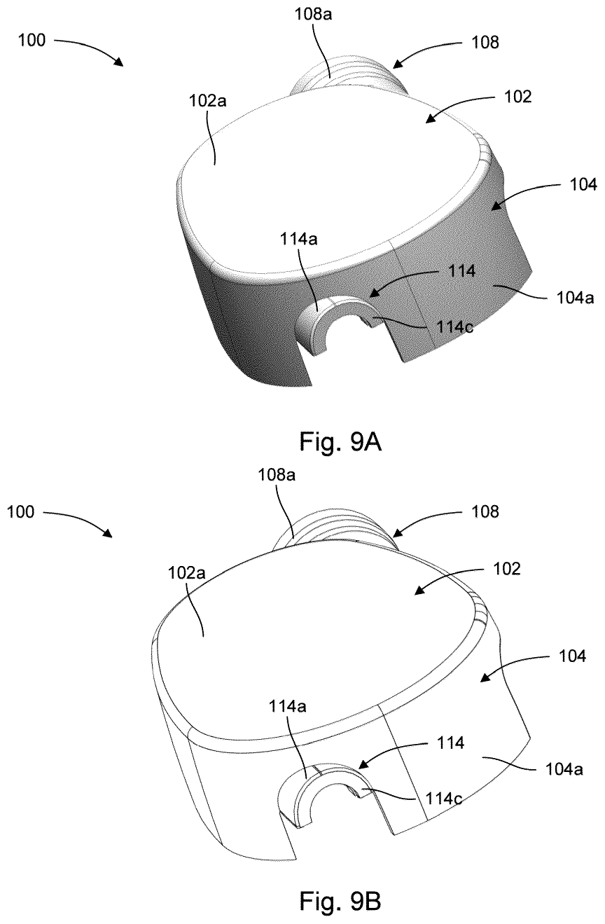

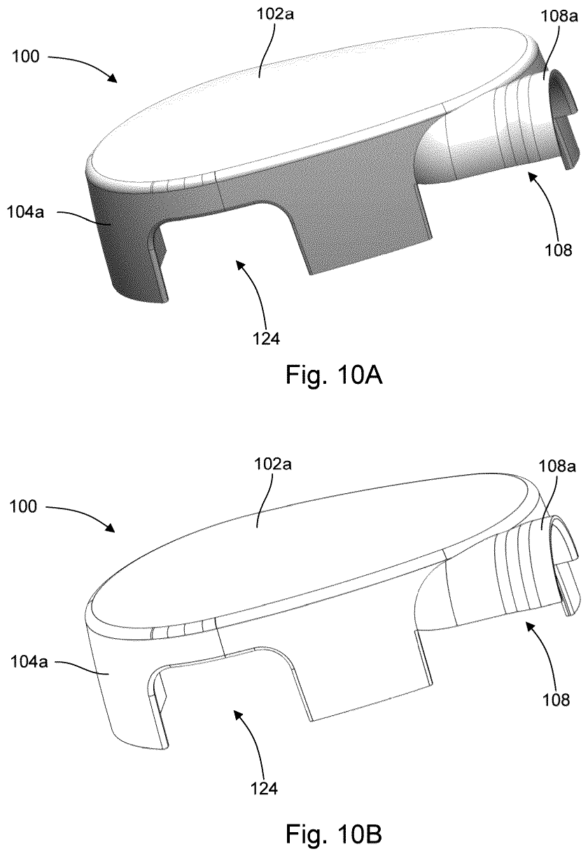

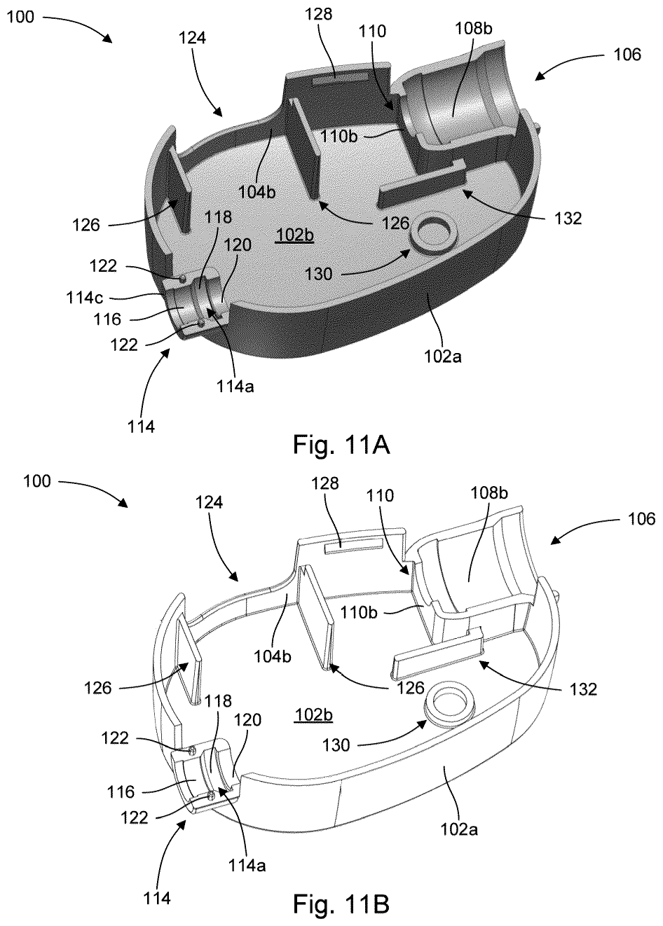

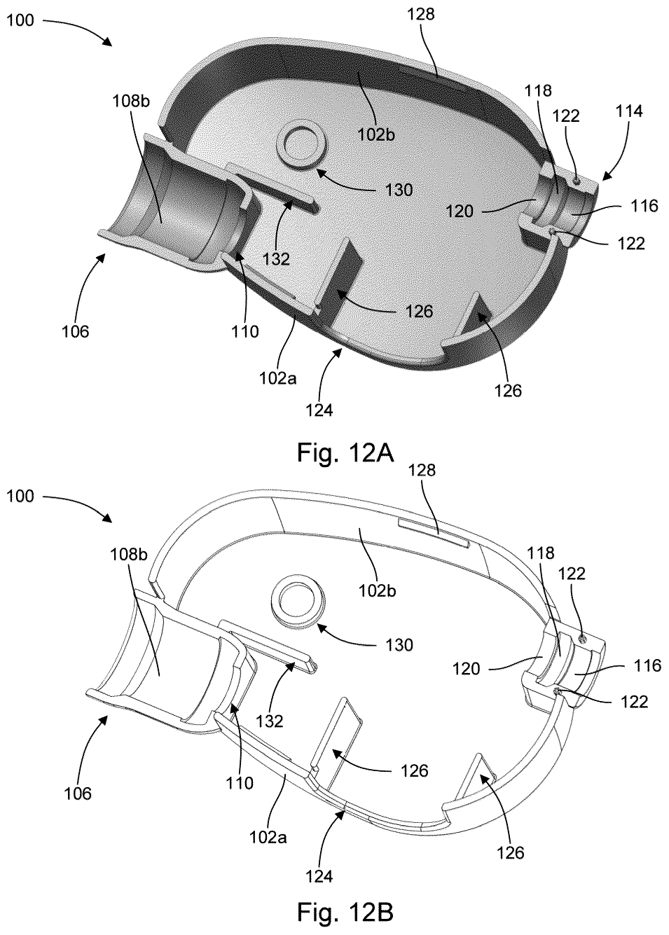

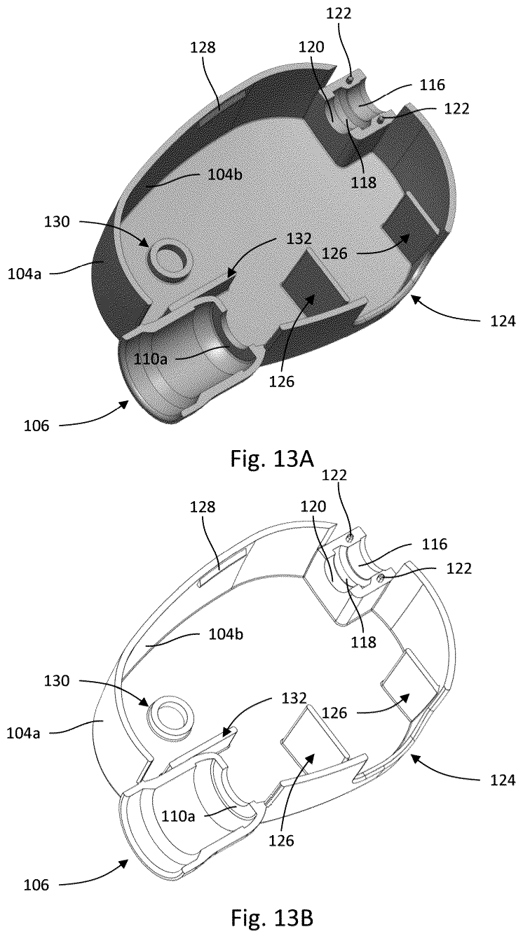

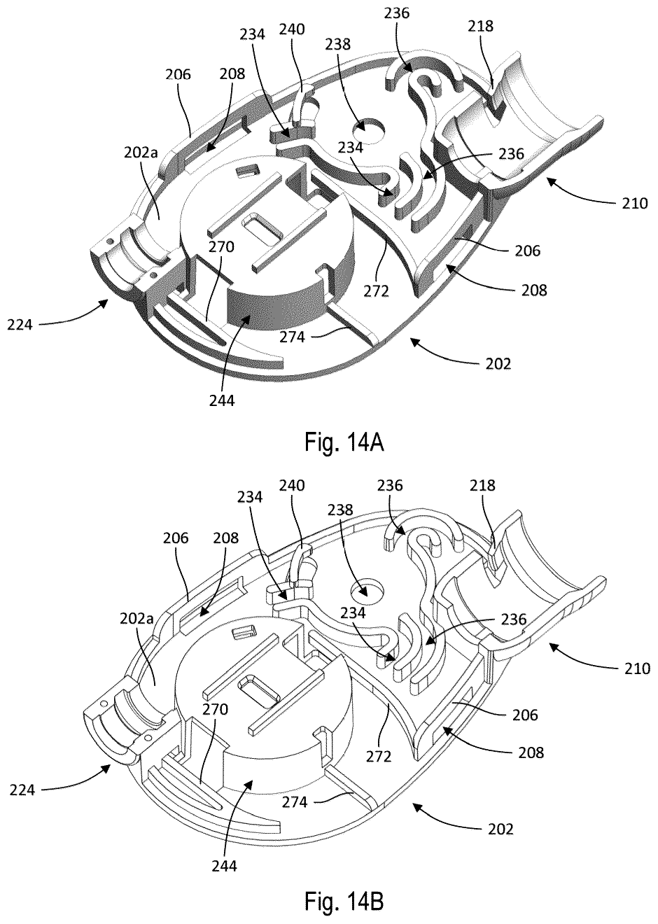

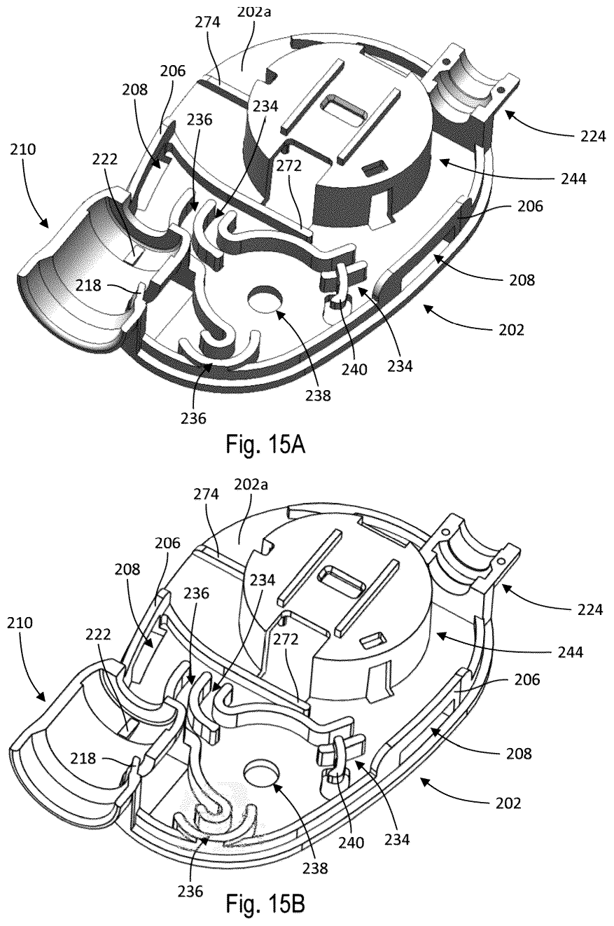

With particular reference to FIGS. 7A and 7B-13A and 13B, the cover 100 is shown in accordance with an exemplary embodiment.

In this embodiment, the cover 100 includes a top wall 102 and a side wall 104. The side wall 104 extends vertically from and perpendicular to the top wall 102. The top wall 102 extends longitudinally from and perpendicular to the side wall 104. The top wall 102 includes an outer surface 102a and an opposed inner surface 102b. The side wall 104 includes an outer surface 104a and an opposed inner surface 104b.

The cover 100 includes a first vial guide 106 which includes an outer wall 108 and an inner end wall 110. The outer wall 108 includes an outer surface 108a and an opposed inner surface 108b. The end wall 110 includes an outer surface 110a and opposed inner surface 110b. The outer wall 108 extends longitudinally from and perpendicular to the outer surface 104a of the side wall 104 and the outer surface 110a of the end wall 110. At least a portion of the inner surface 108b of outer wall 108 of the vial guide 106 has a similar shape and dimension as the outer surface of the vial 14 such that a portion of the outer surface of the vial 14 contacts the inner surface 108b. The end wall 110 defines a U-shaped opening 112.

The cover 100 further includes a first handle retention element 114 which includes an outer surface 114a and an inner surface 114b. The outer surface 114a of the first handle retention element 114 extends from and perpendicular to the outer surface 104a of the side wall 104. The inner surface 114b of the first handle retention element 114 includes a first groove 116, a second groove 118, and a third groove 120. Furthermore, the first handle retention element 114 extends vertically from and perpendicular to the inner surface 102b of the top wall 102. The first handle retention element 114 also includes protrusions 122. As will be discussed in further detail herein, the protrusions 122 aid in coupling the cover 100 to the base 200.

The cover 100 also includes a retraction button opening 124 and retraction button guides 126. The retraction button opening 124 extends through the side wall 104. That is, the retraction button opening 124 extends between the outer surface 104a and the inner surface 104b of the side wall 104. The retraction button guides 126 extend longitudinally from and perpendicular to the inner surface 104b of the side wall 104 and extend vertically from and perpendicular to the inner surface 102b of the top wall 102. The retraction button guides 126 are aligned with the retraction button opening 124 within the cover 100.

The cover 100 further includes a plurality of locking members 128 that extend longitudinally from and perpendicular to the inner surface 104b of the side wall 104. As will be discussed in further detail herein, the locking members 128 couple the cover 100 to the base 200. The cover 100 also includes a circular retention member 130 and a latch guide 132. The circular retention member 130 and the latch guide 132 extend vertically from and perpendicular to the inner surface 102b of the top wall 102.

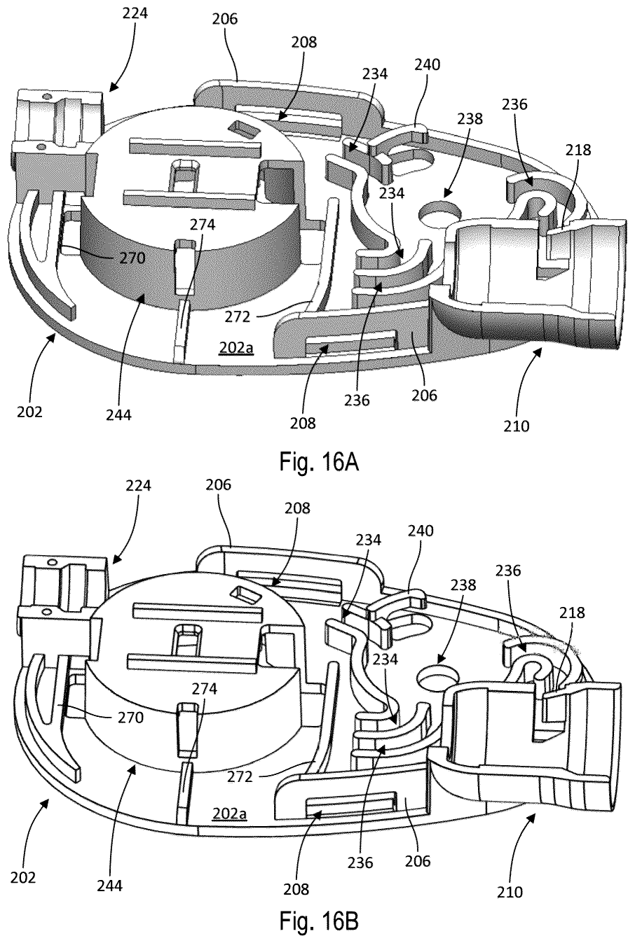

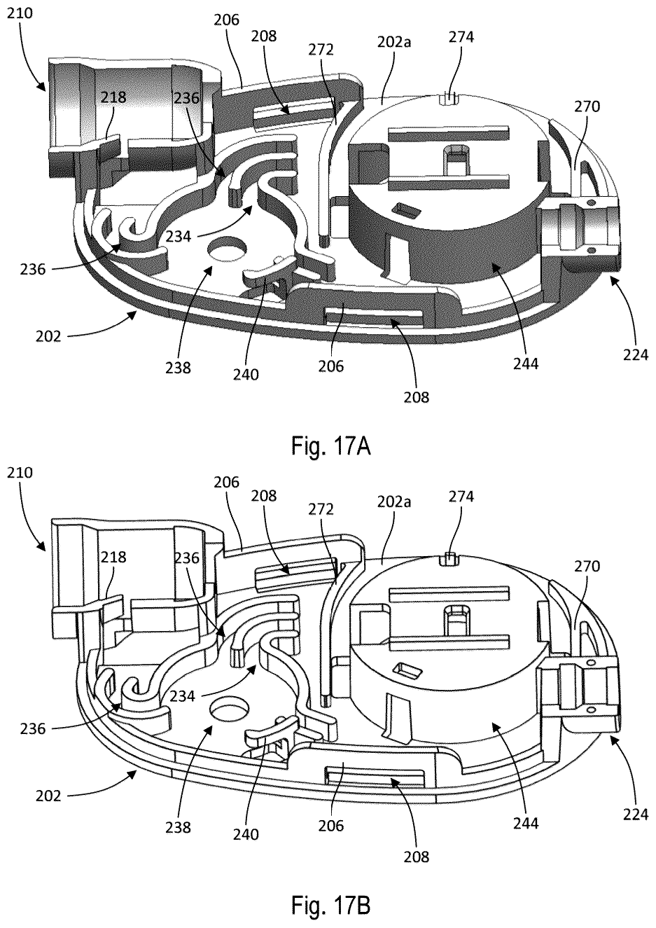

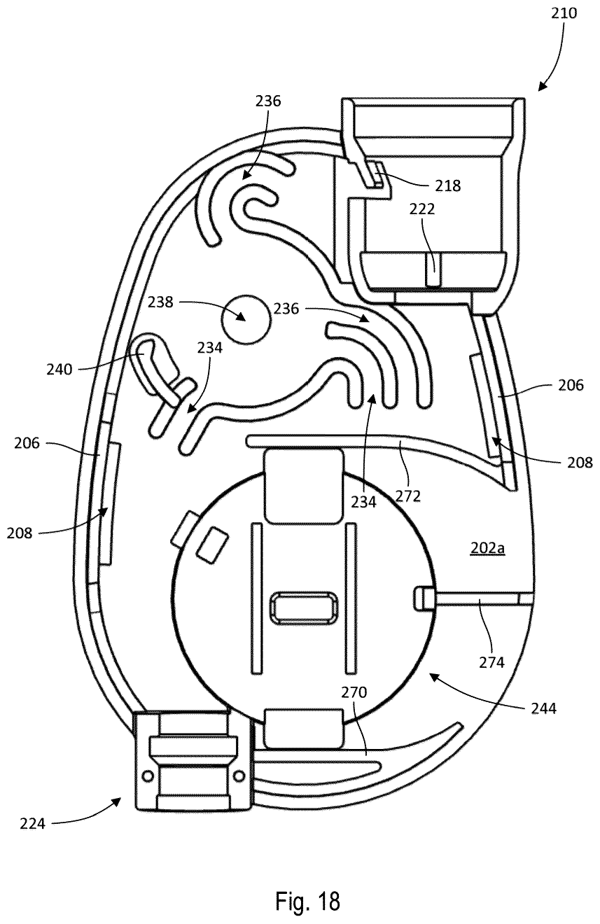

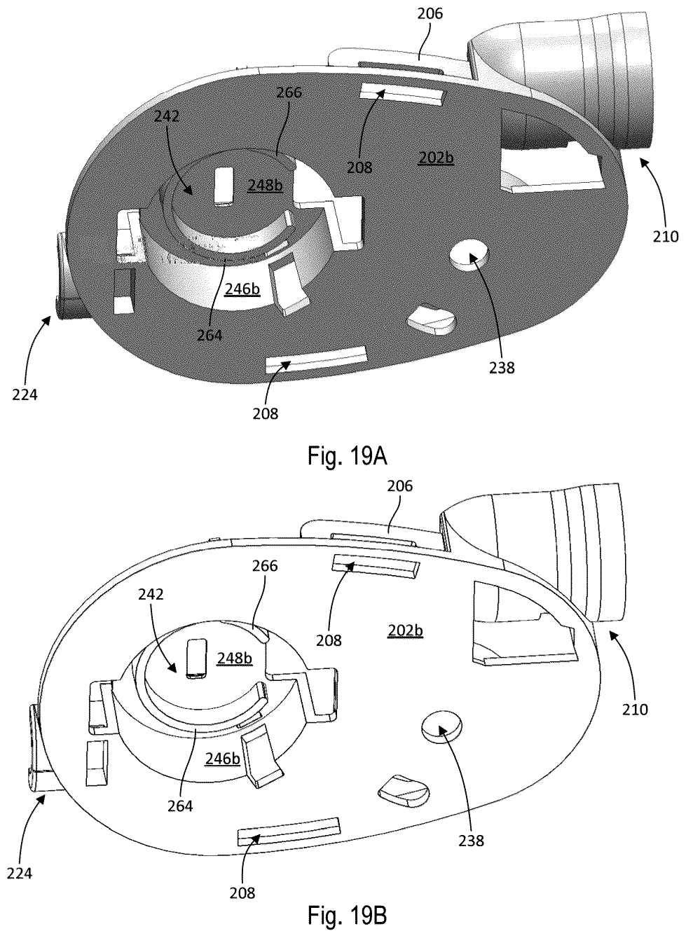

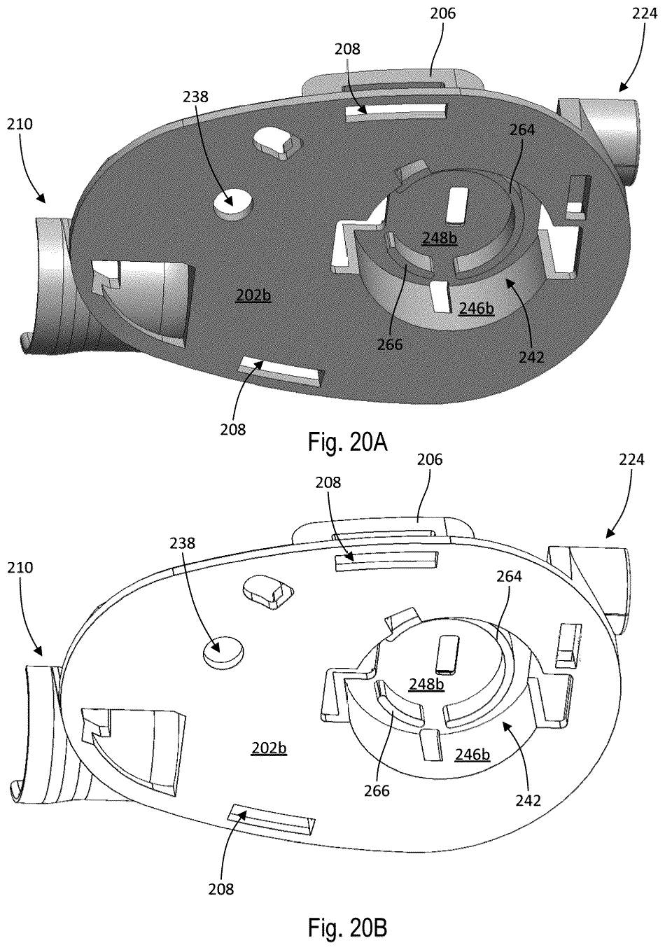

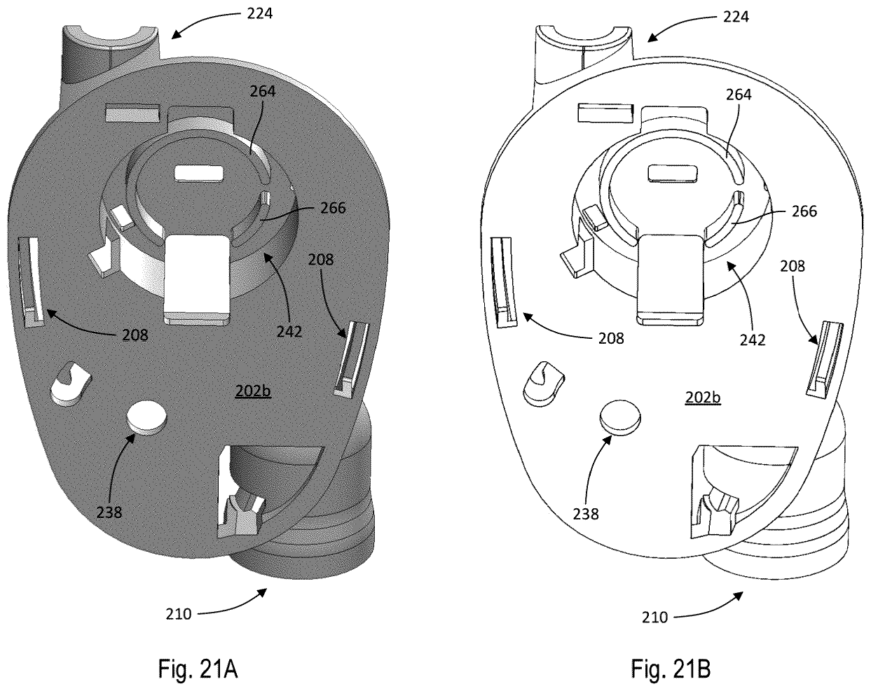

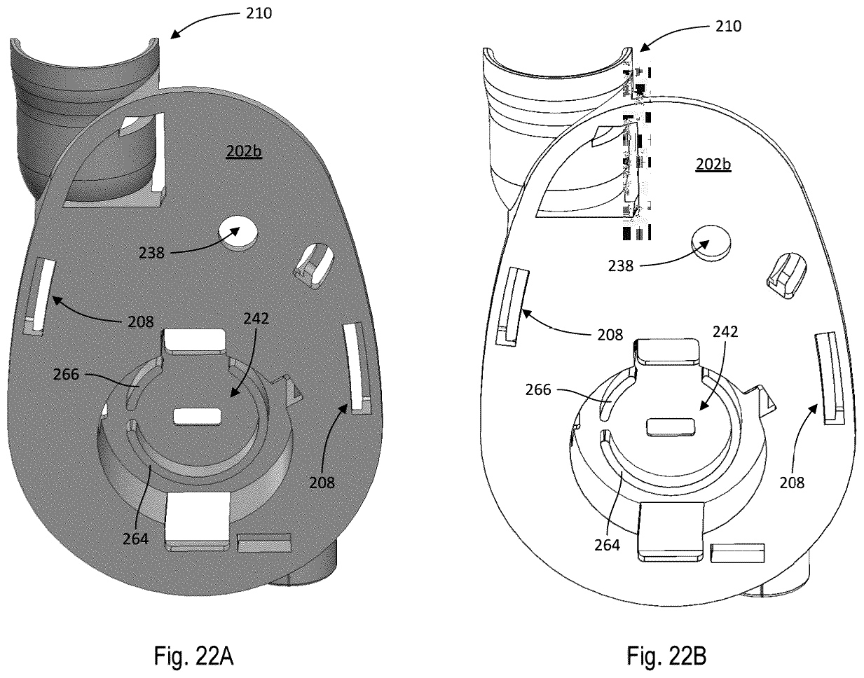

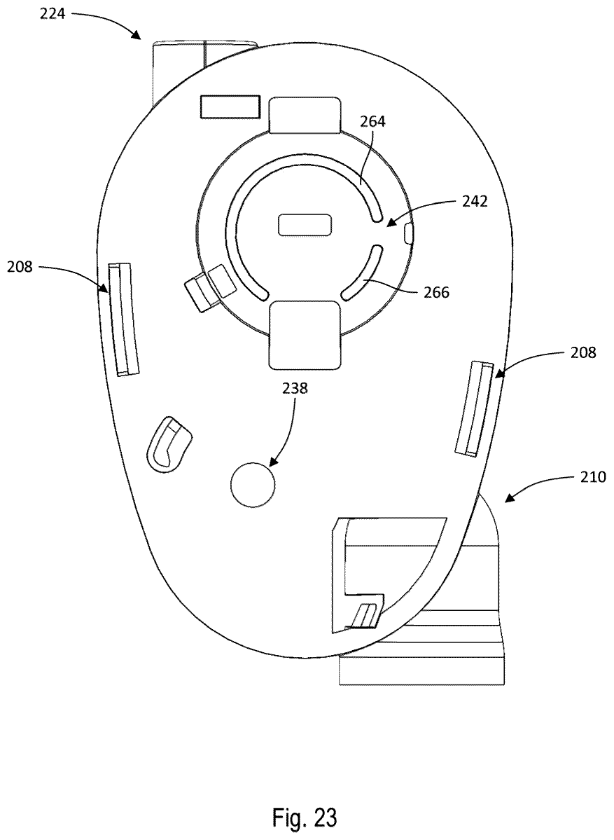

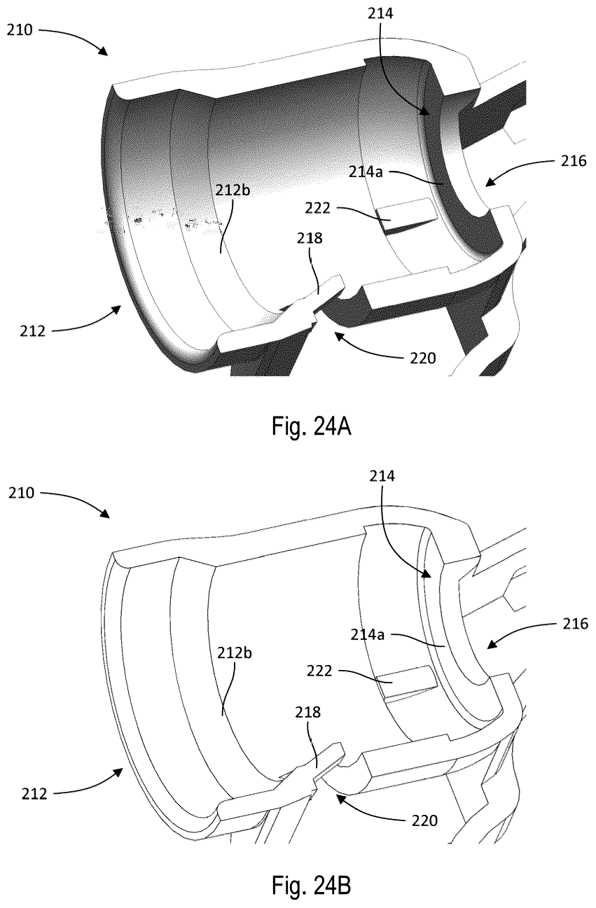

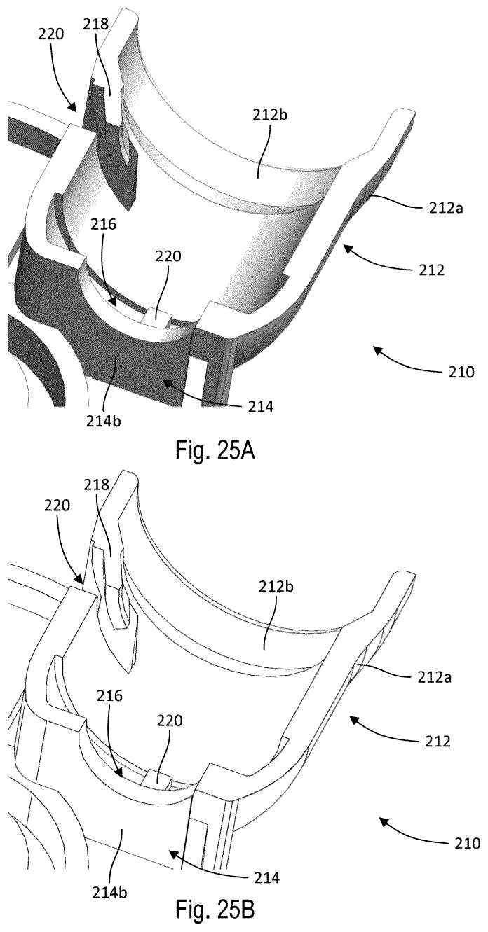

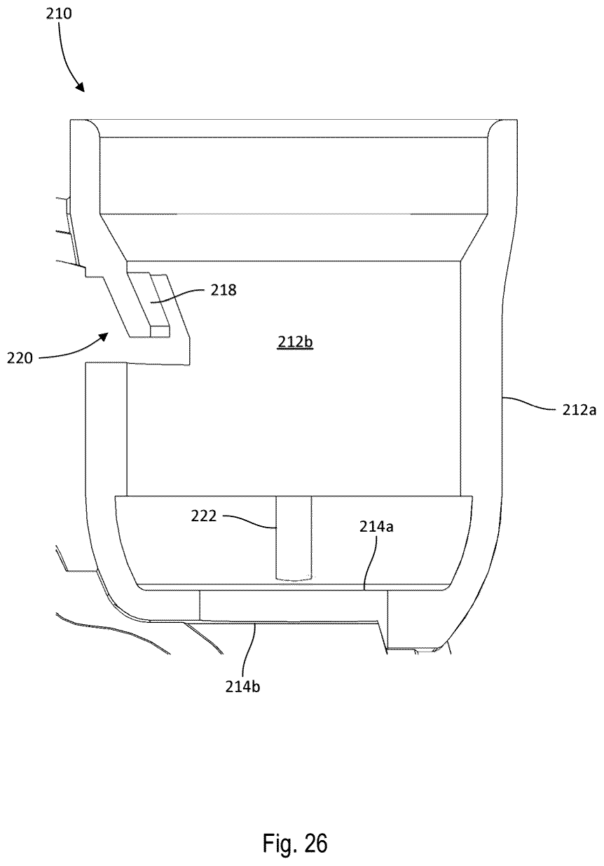

Referring now to FIGS. 14A and 14B-23, the base 200 is shown in accordance with an exemplary embodiment.

The base 200 includes a bottom wall 202 with a top surface 202a and an opposed bottom surface 202b. The bottom wall 202 and the side wall 104 have the same perimeter shape such that when the cover 100 is coupled to the base 200, the cover 100 is flush with the base 200. Furthermore, when the cover 100 is coupled to the base 200 the side wall 104 of the cover 100 contacts the top surface 202a of the bottom wall 202. The base 200 further includes a plurality of extensions 206 that extend vertically from and perpendicular to the top surface 202a of the bottom wall 202. The extensions 206 and the bottom wall 202 define gaps 208. These gaps, and therefore the extensions 206, are shaped and dimensioned to accept a locking member 128 of the cover 100 such that an extension 206 couples to a locking member 128 via a snap fitting. The gaps 208 extend through the bottom wall 202 which provides access to an inner volume of the cartridge 12.

The base 200 further includes a second vial guide 210. The second vial guide 210 includes an outer wall 212 and an inner end wall 214. The outer wall 212 includes an outer surface 212a and an opposed inner surface 212b. The end wall 214 includes an outer surface 214a and an opposed inner surface 214b. At least a portion of the inner surface 212b of the outer wall 212 has a similar shape and dimension to the outer surface of the vial 14 such that a portion of the outer surface of the vial 14 contacts the inner surface 212b of the outer wall 212. The end wall 214 defines a U-shaped opening 216.

The second vial guide 210 also includes a latch 218, a gap 220, and a protrusion 222. The latch 218 extends longitudinally from the outer wall 212 towards a center of the second vial guide 210. The gap 220 is defined by the outer wall 212. The latch 218 extends at least partially across the gap 220. The protrusion 222 extends vertically from the inner surface 212b of the outer wall 212 and extends longitudinally from the outer surface 214a of the end wall 214.

The first vial guide 106 and the second vial guide 210 have a similar shape and dimension and align with one another when the cover 100 is coupled to the base 200. Together, the first vial guide 106 and the second vial guide 210 are referred to as a vial receptacle. Similarly, the end walls 110 and 214 have a similar shape and dimension such that when the cover 100 is coupled to the base 200 the end walls 110 and 214 align and the openings 112 and 216 together define an aperture that provides access to the inner volume of the cartridge 12. Furthermore, the outer surface 108a of the wall 108 of the first vial guide 106 and the outer surface 212a of the outer wall 212 of the second vial guide 210 have a similar shape and dimension such that when the cover 100 is coupled to the base 200, the outer walls 108 and 212 form a uniform cylinder that extends outwardly from the cartridge 12. The inner surface 108b of the outer wall 108 and the inner surface 212b of the outer wall 212 also have a similar shape and dimension such that the inner surfaces 108b and 212b align when the cover 100 is coupled to the base 200. Together, the inner surfaces 108b and 212b with the outer surfaces 110a and 214a define an inner chamber of the vial receptacle.

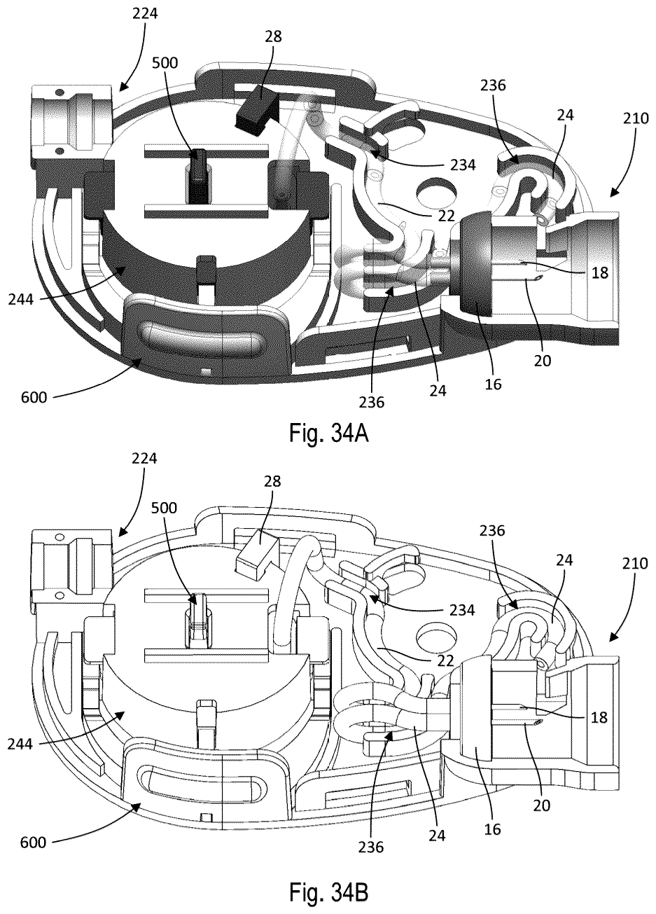

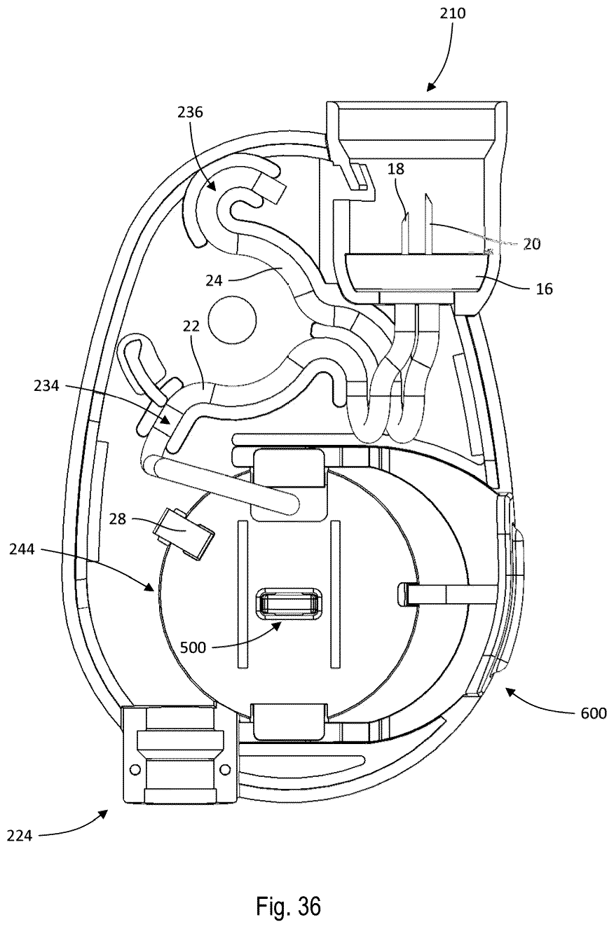

With particular reference to FIGS. 34A and 34B-36, the cartridge 12 includes a stop 16 that is generally cylindrical in shape. The stop 16 may be formed of a polymeric or an elastomeric material. When the cover 100 is coupled to the base 200, the stop 16 is disposed within the inner volume of the vial receptacle and is held in place by the protrusion 222. A portion of the inner surface 108b of the outer wall 108 and a portion of the inner surface 212b of the outer wall 212 has a similar shape and dimension as the outer surface of the stop 16 such that the inner surfaces 108b and 212b retain the stop 16 within the vial receptacle. That is, when the stop 16 is disposed within the inner volume of the vial receptacle, the stop 16 contacts portions of the inner surfaces 108b and 212b and contacts the outer surfaces 110a and 214a of the end walls 110 and 214 respectively. Furthermore, when the cover 100 is coupled to the base 200, a portion of the stop 16 extends through the aperture defined by the end walls 110 and 214.

In some embodiments, the cartridge 12 includes a first hollow needle 18 and a second hollow needle 20 that extend through the stop 16. When the vial 14 is inserted into the vial receptacle defined by the walls 108 and 212, the needles 18 and 20 pierce a cap of the vial 14 to provide access to a pharmaceutical stored therein. Furthermore, while the vial 14 is being inserted into the vial receptacle, the latch 218 is compressed into the gap 220m which allows the vial 14 to extend into the vial receptacle until the end of the vial 14 contacts the stop 16. When the vial 14 is completely inserted, the latch 218 returns to its original position. In this position, when the vial 14 is pulled, the cap of the vial 14 contacts the latch 218 which prevents the vial 14 from being removed from the cartridge 12.

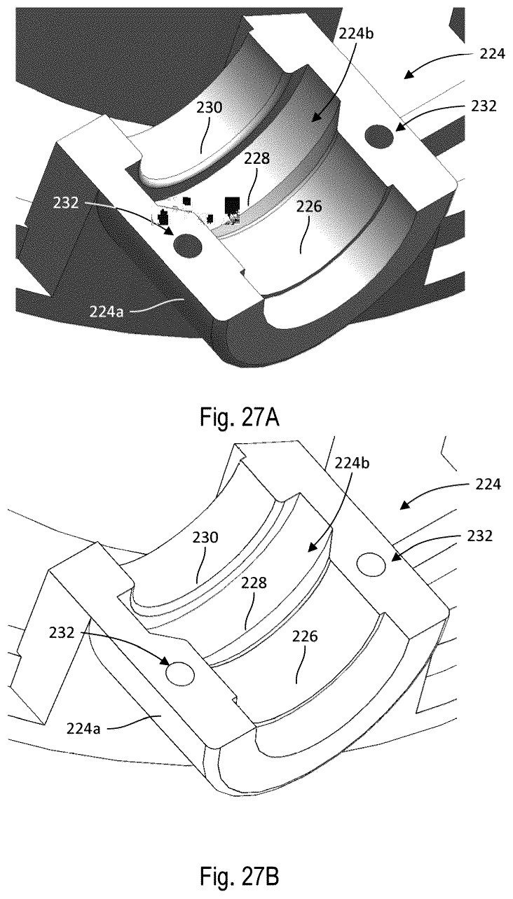

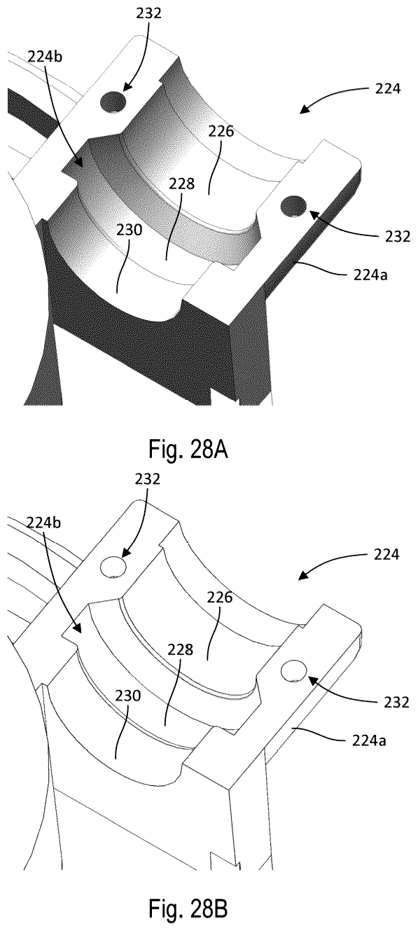

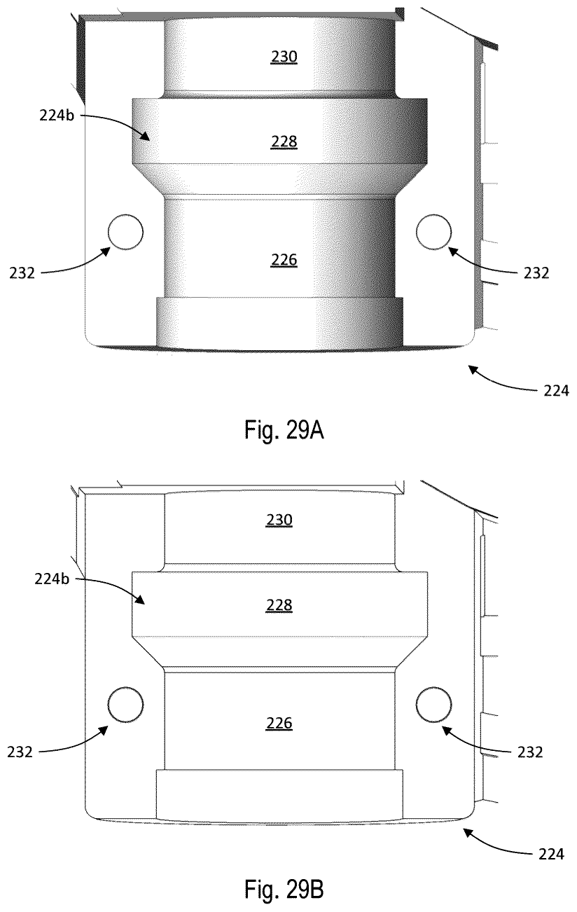

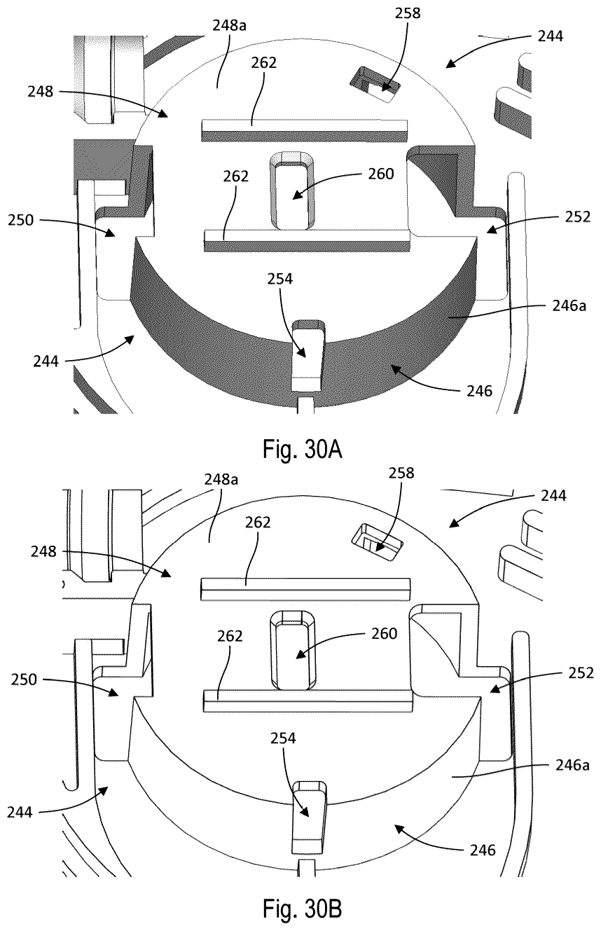

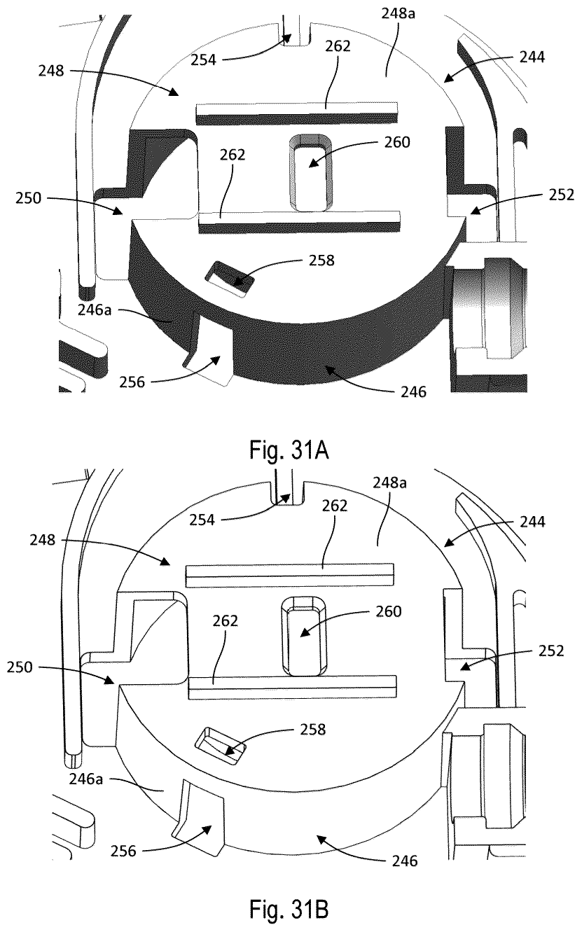

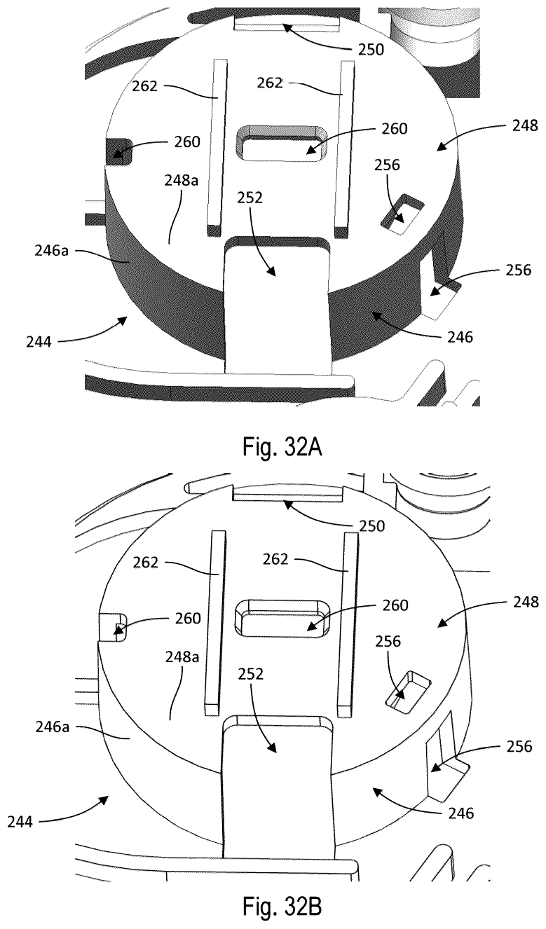

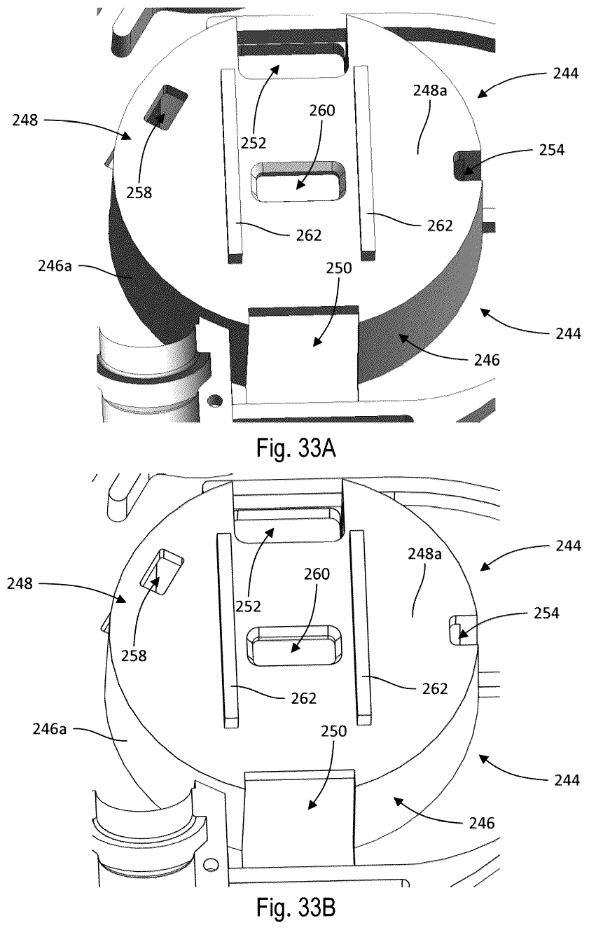

With continued reference to FIGS. 27A and 27B-29A and 29B, the base 200 further includes a second handle retention element 224. second handle retention element 224 includes an outer surface 224a and an opposed inner surface 224b. The inner surface 224b of the second handle retention element 224 includes a first groove 226, a second groove 228, and a third groove 230. Furthermore, the second handle retention element 224 extends vertically from and perpendicular to the top surface 202a of the bottom wall 202. The second handle retention element 224 includes cavities 232. When the cover 100 is coupled to the base 200, the protrusions 122 extend into the cavities 232 to couple the cover 100 to the base 200.

The first handle retention element 114 and the second handle retention element 224 have a similar shape and dimension and align with one another when the cover 100 is coupled to the base 200. Together, the first handle retention element 114 and the second handle retention element 224 are referred to as a handle receptacle. Furthermore, when the cover 100 is coupled to the base 200, the first handle retention element 114 and the second handle retention element 224 align such that the handle receptacle has an outer opening and inner opening which allows at least a portion of the pull mechanism to extend through the handle receptacle. The outer surface 114a of the first handle retention element 114 and the outer surface 224a of the second handle retention element 224 have a similar shape and dimension such that when the cover 100 is coupled to the base 200, the outer surfaces 114a and 224a form a uniform cylinder that extends outwardly from the cartridge 12. The inner surface 114b and the inner surface 224b also have a similar shape and dimension such that the inner surfaces 114b and 224b align when the cover 100 is coupled to the base 200. Together, the inner surfaces 114b and 224b define an inner chamber of the handle receptacle.

Furthermore, the first grooves 116 and 226 have a similar shape and dimension, the second grooves 118 and 228 have a similar shape and dimension, and the third grooves 120 and 230 have a similar shape and dimension. When the cover 100 is coupled to the base 200, the first grooves 116 and 226 align, the second grooves 118 and 228 align and the third grooves 120 and 230 align. Together, the first grooves 116 and 226 are referred to as a first handle retention groove, the second grooves 118 and 228 are referred to as a ball retention groove, and the third grooves 120 and 230 are referred to as a second handle retention groove.

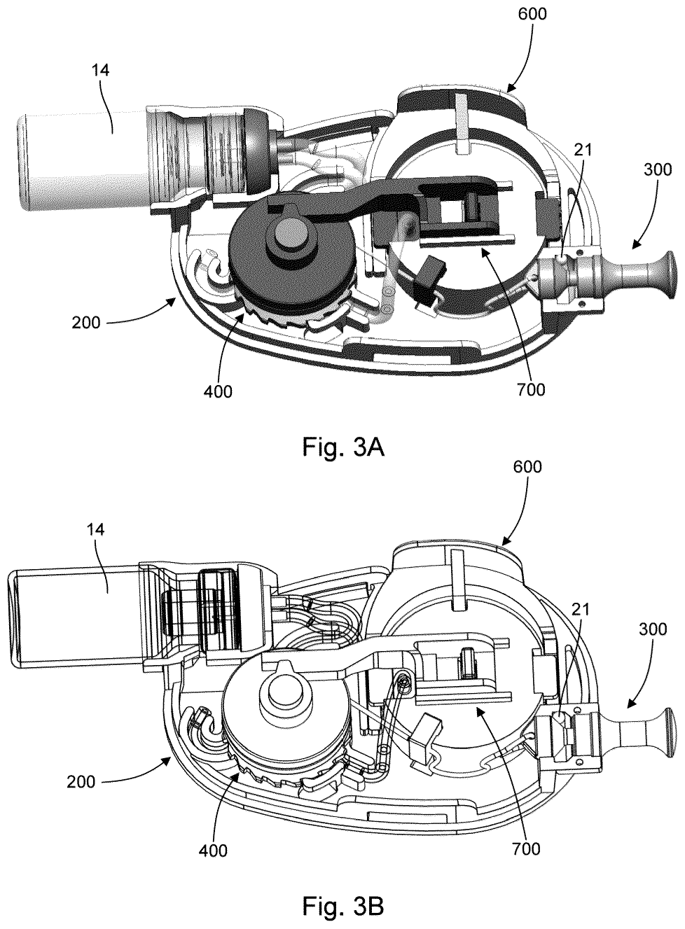

The base 200 further includes a first tube guide 234 and a second tube guide 236 that extend vertically from and the top surface 202a of the bottom wall 202. In some embodiments, as depicted in FIGS. 34A and 34B-36, the cartridge 12 includes a first tube 22 and a second tube 24. In these embodiments, the first tube guide 234 retains the first tube 22 and the second tube guide 236 retains the second tube 24 within the cartridge 10. The tube guides 234 and 236 direct the tubes 22 and 24 around a portion of the pump assembly 400 (FIGS. 3A and 3B). Furthermore, the first tube guide 234 directs the first tube 22 towards the microneedle array assembly 500. The first tube 22 and the second tube 24 fit around the first needle 18 and the second needle 20 respectively. As such, the tubes 22 and 24 are in open communication with the vial 14 via the needles 18 and 20. In other embodiments, the second tube 24 may be omitted. In these embodiments, air may enter the vial 14 via the second needle 20.

With continued reference to FIGS. 14A and 14B-23, the base 200 further includes retention aperture 238 that extends through the bottom wall 202. Stated another way, the retention aperture 238 extends between the top surface 202a and the bottom surface 202b of the bottom wall 202. When the cover 100 is coupled to the base 200, the retention aperture 238 is disposed vertically below the circular retention member 130. As will be discussed in further detail herein, the circular retention member 130 and the retention aperture 238 aid in coupling the pump assembly to the cover 100 and the base 200.

The base 200 also includes a latch 240 that extends vertically from and perpendicular to a portion of the first tube guide 234. As will be discussed in further detail herein, the latch 240 allows the pump to pump the pharmaceutical from the vial and to the microneedle assembly 400. The latch 240 allows the pump assembly 400 to rotate in a clockwise direction and prevents the pump assembly 400 from rotating in a counterclockwise direction. Rotating in the counterclockwise direction would result in the pump assembly 400 failing to pump the pharmaceutical.

The base 200 further includes a microneedle array aperture 242 and a microneedle array housing 244. The microneedle array aperture 242 extends through the bottom wall 202. That is, the microneedle array aperture 242 extends between the top surface 202a and the bottom surface 202b of the bottom wall 202. The microneedle array aperture 242 is shaped and dimensioned to allow a portion of the microneedle array assembly 500 to extend through the bottom wall 202.

The microneedle array housing 244 is generally cylindrical in shape and is shaped and dimensioned to house the microneedle array assembly 500. While the microneedle array housing 244 and the microneedle array assembly 500 are depicted as being cylindrical, it is understood that the microneedle array housing 244 and the microneedle array assembly 500 may have a different shape. The microneedle array housing 244 extends vertically from and perpendicular to the top surface 202a of the bottom wall 202. The microneedle array housing 244 includes a side wall 246 and a top wall 248. The side wall 246 extends vertically between and perpendicular to the top surface 202a of the bottom wall 202 and the top wall 248. The top wall 248 extends longitudinally between the side wall 246. The side wall 246 includes an outer surface 246a and an opposed inner surface 246b. The top wall 248 includes an outer surface 248a and an opposed inner surface 248b.

The microneedle array housing 244 and the bottom wall 202 include a first extension opening 250 and a second extension opening 252 that are positioned on opposite sides of the microneedle array housing 244. The extension openings 250 and 252 extend through the walls 246 and 248. That is, the extension openings 250 and 252 extend between the outer surface 246a and the inner surface 246b of the side wall 246, extend between the surfaces 248a and 248b of the top wall 248, and extend between the top surface 202a and the bottom surface 202b of the bottom wall 202. The microneedle array housing 244 further includes an opening 254 that is positioned between the extension openings 250 and 252. The opening 254 extends through the side wall 246 and the top wall 248. That is, the opening 254 extends between the surfaces 246a and 246b of the side wall 246 and extends between the surfaces 248a and 248b of the top wall 248.

The microneedle array housing 244 also includes a first tensioner opening 256 and a second tensioner opening 258 that are aligned with one another. A portion of the first tensioner opening 256 extends through the side wall 246 and another portion of the first tensioner opening 256 extends through the bottom wall 202. That is, the first tensioner opening 256 extends between the outer surface 246a and the inner surface 246b of the side wall 246 and extends between the top surface 202a and the bottom surface 202b of the bottom wall 202. The second tensioner opening 258 extends through the top wall 248 of the microneedle array housing 244. That is, the second tensioner opening 258 extends between the outer surface 248a and the inner surface 248b of the top wall 248.

The top wall 248 of the microneedle array housing 244 also includes a microneedle array assembly aperture 260 that extends through the top wall 248 of the microneedle array housing 244. That is, the microneedle array assembly aperture 260 extends between the outer surface 246a and the inner surface 246b of the wall 246. The top wall 248 of the microneedle array housing 244 also includes a first and second trigger guide 262. The trigger guides 262 extend vertically from and perpendicular to the outer surface 248a of the top wall 248. The trigger guides 262 are positioned on opposite sides of the microneedle array assembly aperture 260.

The microneedle array housing 244 further includes a first partially circular projection 264 and a second partially circular projection 266 that extend vertically from and perpendicular to the inner surface 248b of the top wall 248.

Referring again to FIGS. 14A and 14B-23, the base 200 includes a first button guide 270 and a second button guide 272 that extend vertically from and perpendicular to the top surface 202a of the bottom wall 202. The first and second button guides 270 and 272 extend along opposite sides of the microneedle array housing 244. Furthermore, the first button guide 270 extends along the first extension opening 250 and the second button guide 272 extends along the second extension opening 252. As will be discussed in further detail herein, the first and second button guides 270 and 272 direct a portion of the retraction button 600 towards the first extension opening 250 and second extension opening 252. The base 200 further includes a third button guide 274. The third button guide 274 extends vertically from and perpendicular to the top surface 202a of the bottom wall 202. The third button guide 274 is disposed between the first and second button guides 270 and 272 and is aligned with the opening 254 of the microneedle array housing 244. As will be discussed in further detail herein, the third button guide 274 directs a portion of the retraction button 600 towards the opening 254.

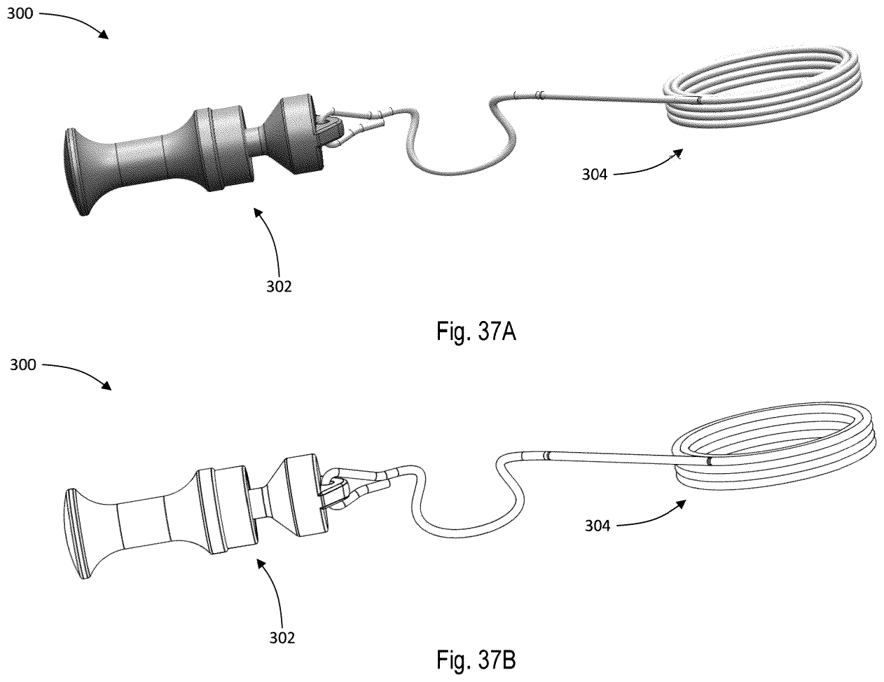

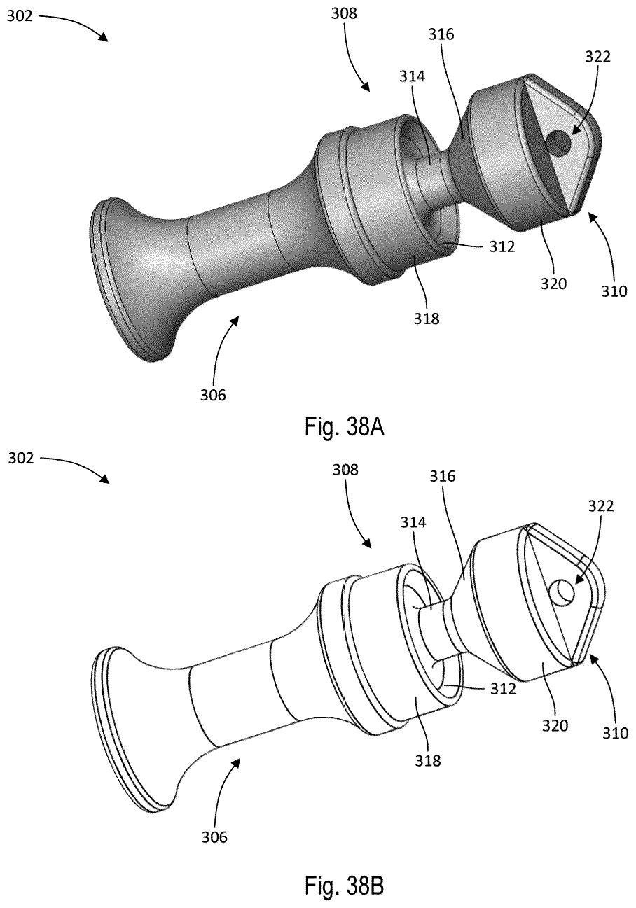

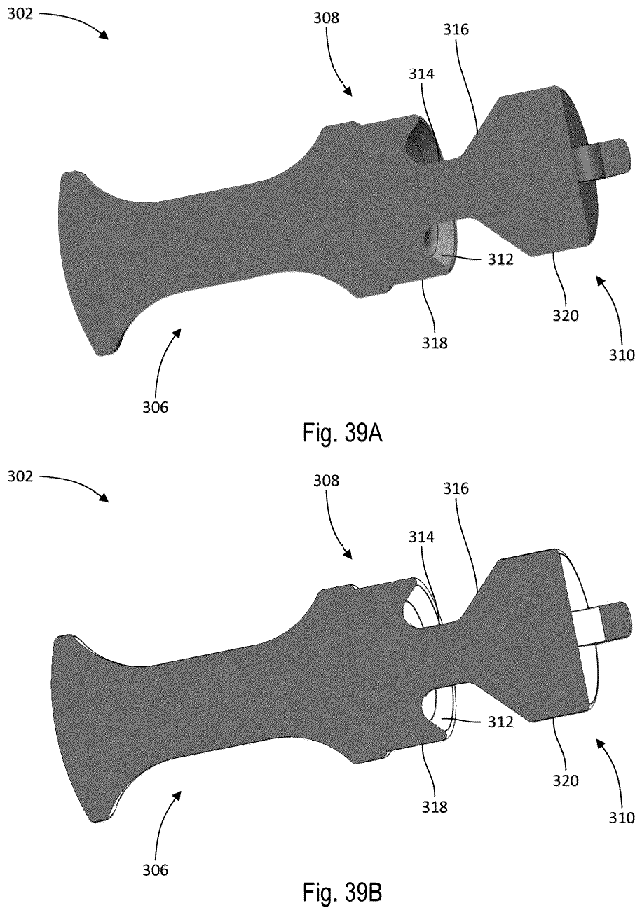

Referring now to FIGS. 37A and 37B-39A and 39B, the pull mechanism 300 is shown in accordance with an exemplary embodiment. The pull mechanism 300 includes a handle 302 and a cord 304 that is connected to the handle 302.

The handle 302 includes a grip 306, a ball retention feature 308, and an anchor portion 310. The grip 306 defines a distal end of the handle 302 and the ball retention feature 308 defines a proximal end of the handle 302.

The ball retention feature 308 includes a rounded inner surface 312, a cylinder 314 and an angled surface 316. As will be discussed in further detail herein, the rounded inner surface 312, the cylinder 314, and the angled surface 316 are shaped and dimensioned to retain a ball 21 of the cartridge 12 when the cartridge 12 is in a given orientation and is shaped and dimensioned to release the ball 21 when the cartridge 12 is in any other orientation. The ball retention feature 308 includes a first outer surface 318 and a second outer surface 320. The outer surfaces 318 and 320 have a similar shape and dimensioned as the first handle retention grove and the second handle retention grove which allows the handle retention features 114 and 224 to retain the handle 302 of the pull mechanism 300.

The anchor portion 310 includes an aperture 322 that extends through the anchor portion 310. The cord 304 extends through the aperture 322 which allows securing the cord 304 to the anchor portion 310.

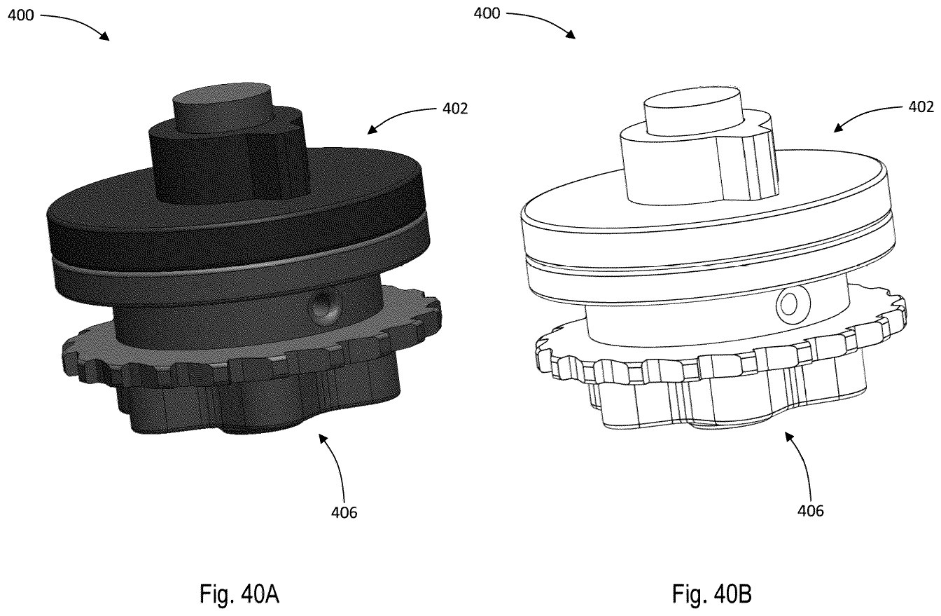

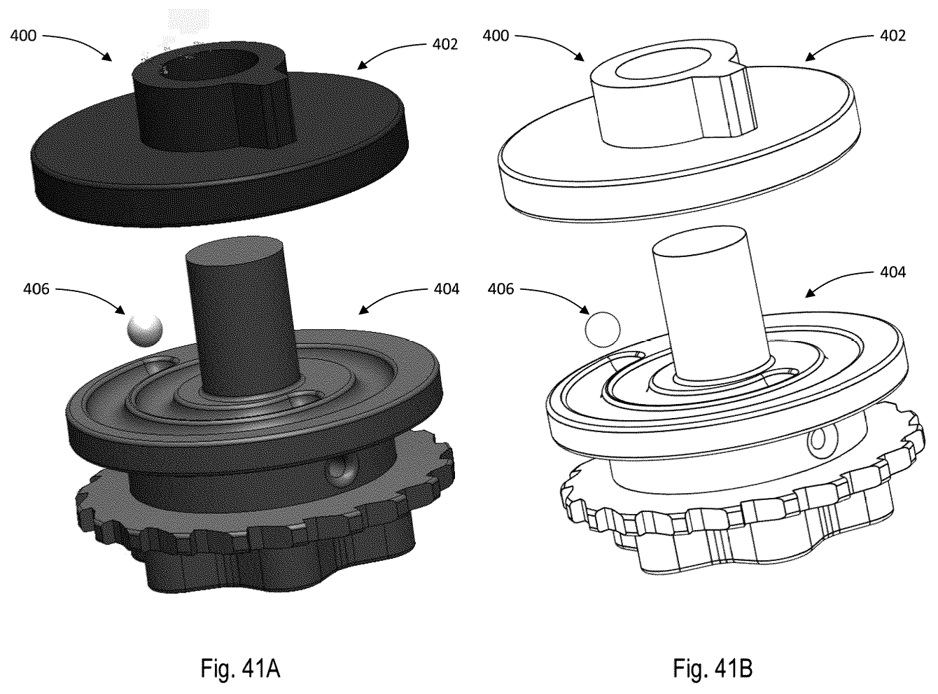

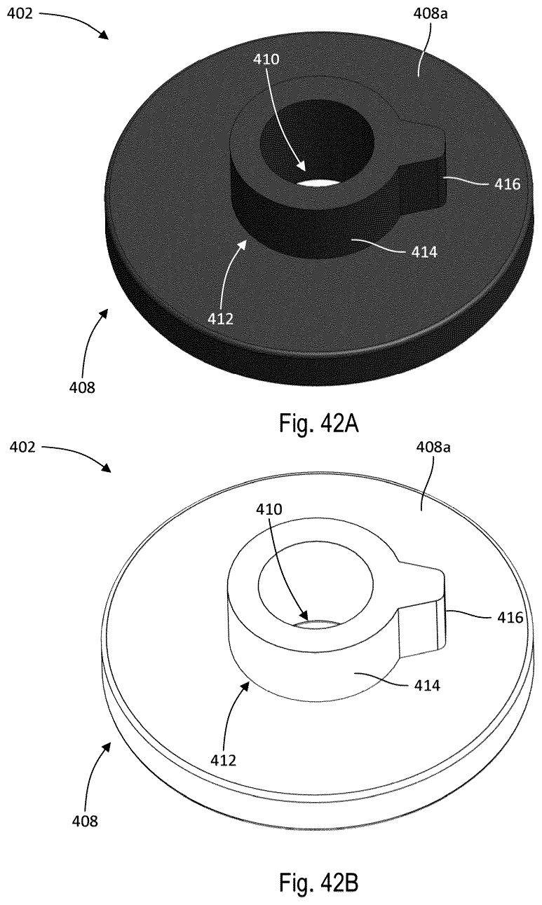

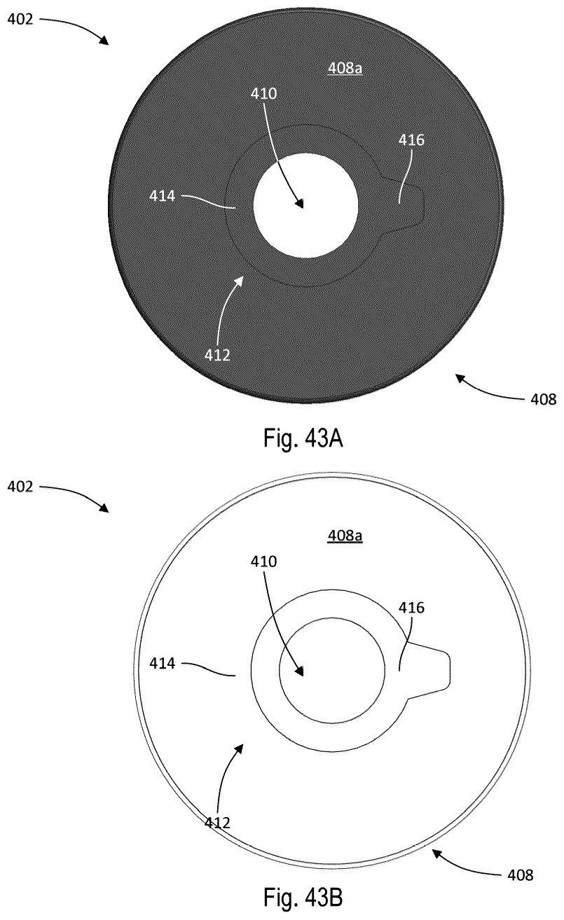

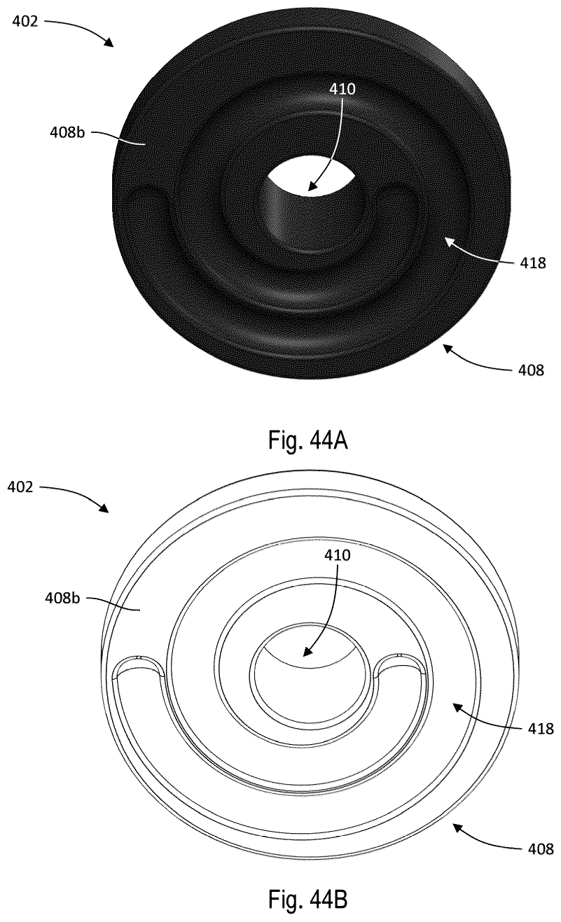

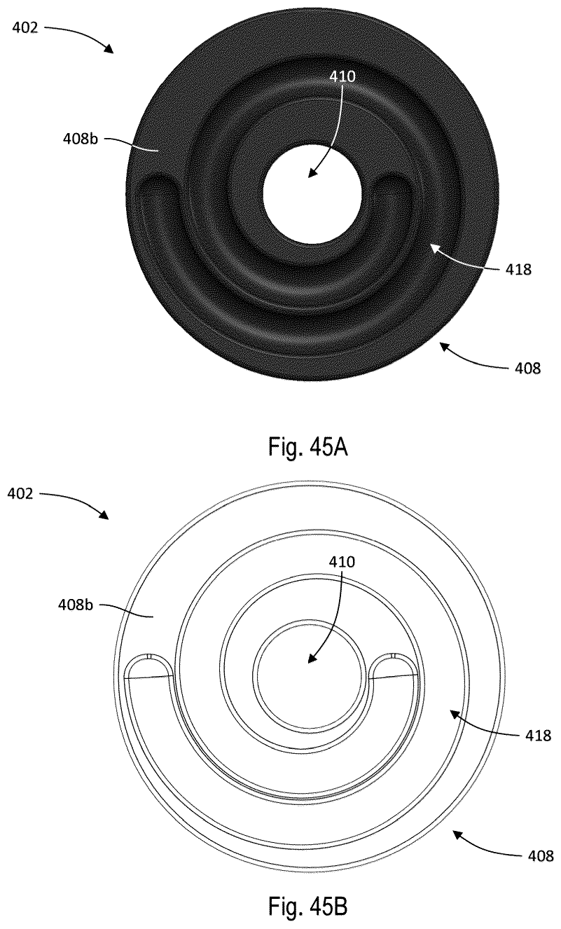

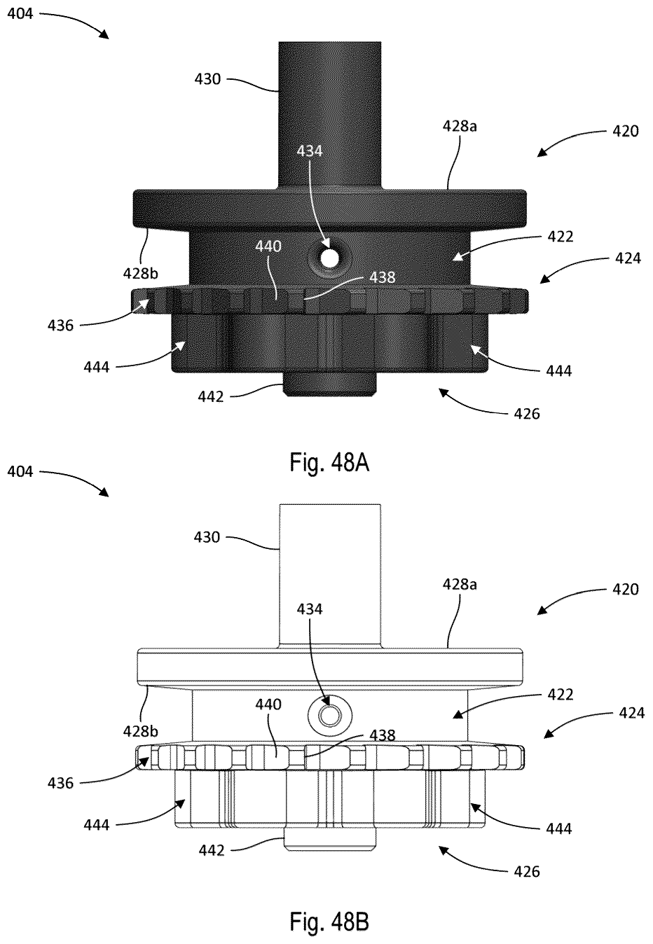

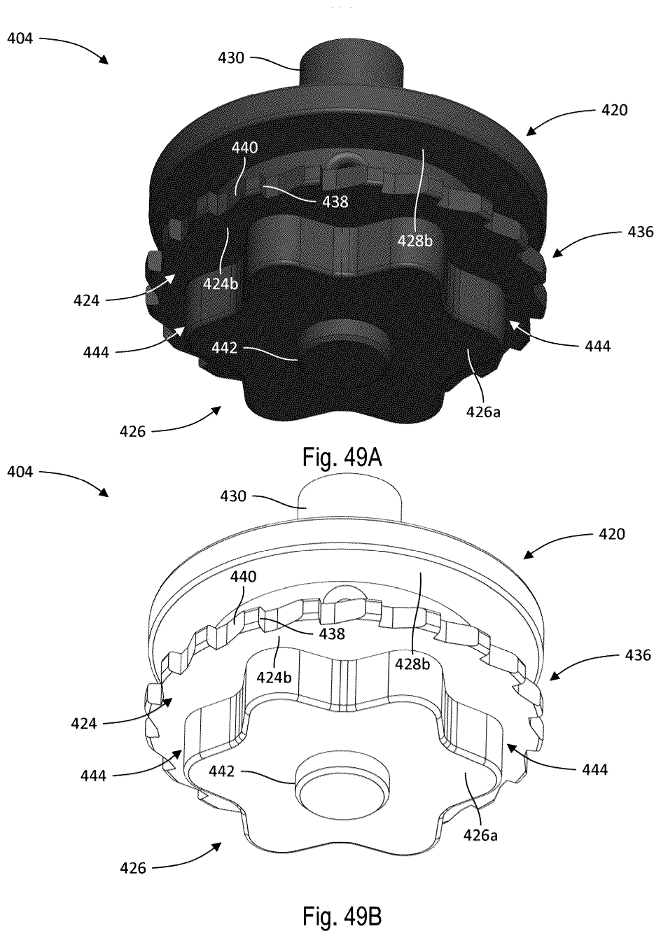

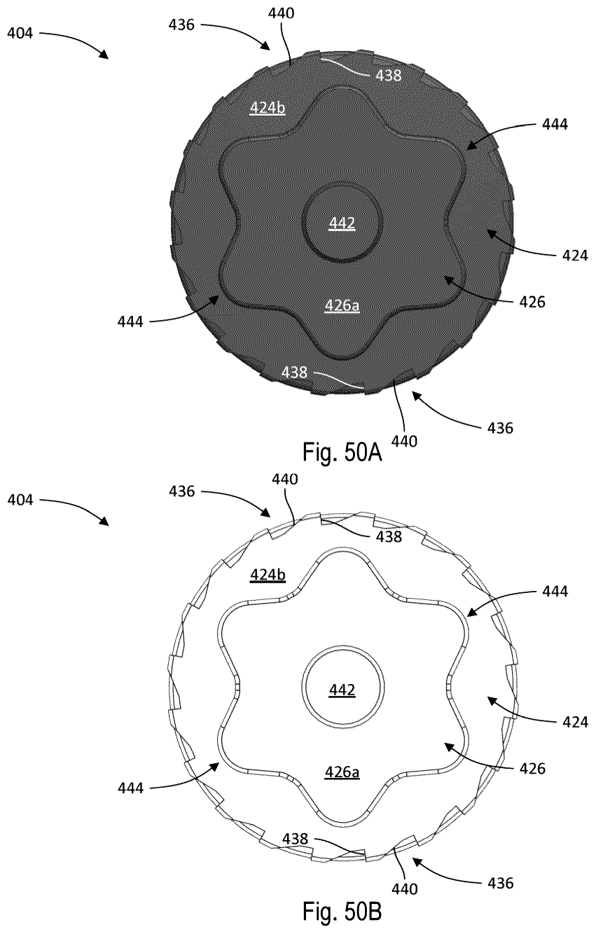

Referring now to FIGS. 40A and 40B-50A and 50B, the pump assembly 400 is shown in accordance with an exemplary embodiment. In this embodiment, the pump assembly 400 includes a trigger portion 402 and a pump portion 404 that are coupled to one another. The pump assembly 400 also includes a ball 406 that is disposed between the trigger portion 402 and the pump portion 404.

The trigger portion 402 includes a base 408 with a top surface 408a and an opposed bottom surface 408b. The base 408 includes an aperture 410 that extends through the base 408. That is, the aperture 410 extends between the top surface 408a and the bottom surface 408b. The trigger portion 402 further includes a latch 412 which extends vertically from and perpendicular to the top surface 408a of the base 408. The latch 412 includes a circular portion 414 and an extension 416. The circular portion extends circumferentially around the aperture 410 and the extension 416 extends longitudinally from and perpendicular to the circular portion 414. The bottom surface 408b of the base 408 defines a spiral groove 418. The spial groove 418 is shaped and dimensioned to retain the ball 406 therein.

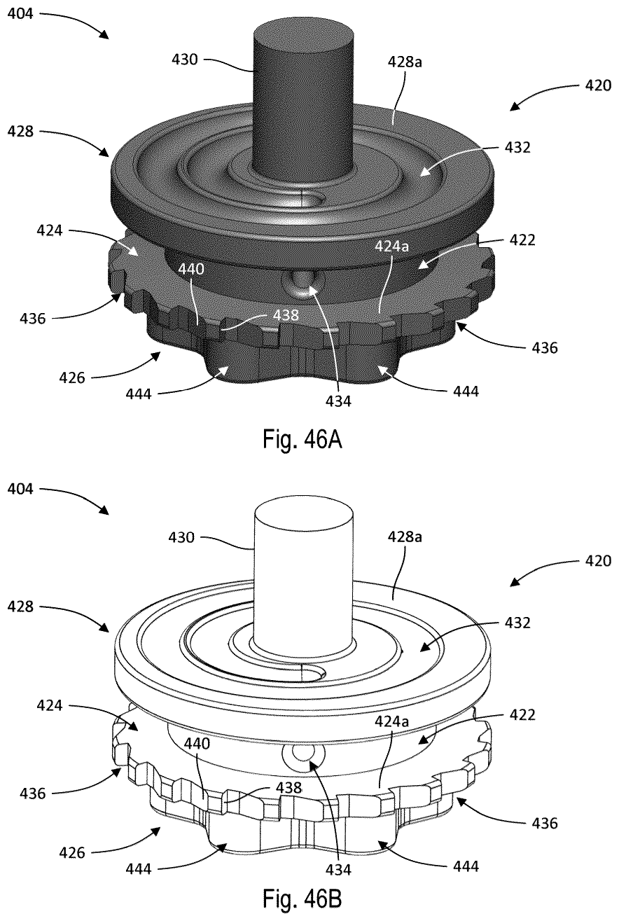

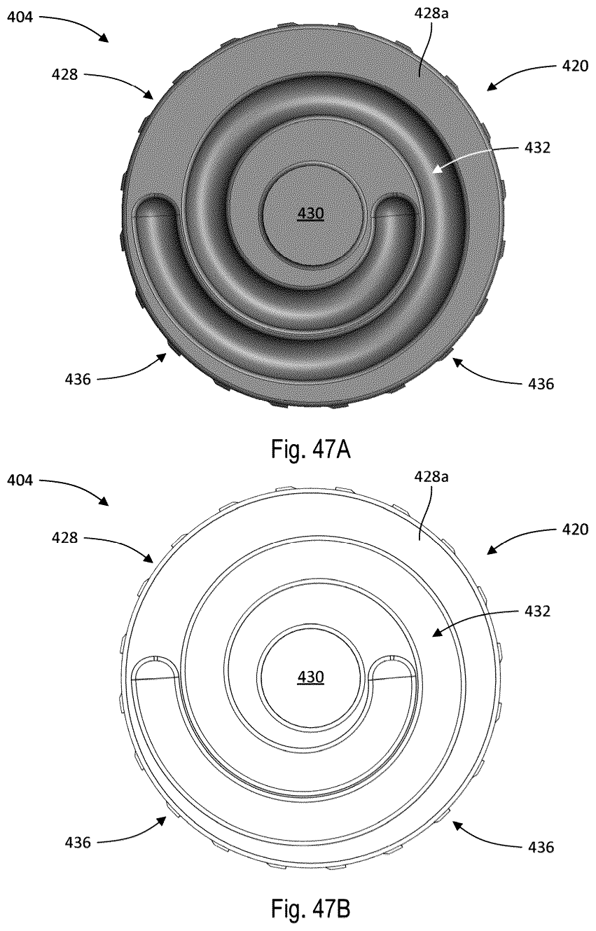

With reference to FIGS. 46A and 46B-50A and 50B, the pump portion 404 includes a top portion 420, a middle support portion 422, a ratchet 424, and a cog 426.

The top portion 420 includes a base 428 with a top surface 428a and an opposed bottom surface 428b. The top portion 420 further includes a top cylinder 430 that extends vertically from and perpendicular to the top surface 428a of the base 428. When the trigger portion 402 is coupled to the pump portion 404, the top cylinder 430 extends through the aperture 410 and beyond the circular portion 414 of the latch 412. Furthermore, when the pump assembly 400 is disposed within the cartridge 12, the top cylinder 430 extends into the circular retention member 130 of the cover 100 which aids in coupling the pump assembly 400 to the cover 100. As will be discussed in further detail herein, the circular retention member 130 maintains the pump assembly 400 in a given position while allowing the pump assembly 400 to rotate.

The top surface 428a of the base 428 defines a spiral groove 432 that is shaped and dimensioned to retain the ball 406 therein. The base 408 of the trigger portion 402 has a similar shape and dimension as the base 428. Furthermore, the spiral groove 418 of the base 408 and the spiral groove 432 of the base 428 have a similar shape and dimension and when the trigger portion 402 is coupled to the pump portion 404, the spiral grooves 418 and 432 align with one another and retain the ball 406 therebetween. Together, the spiral groove 418 and the spiral groove 432 are referred to as a spiral ball retention groove.

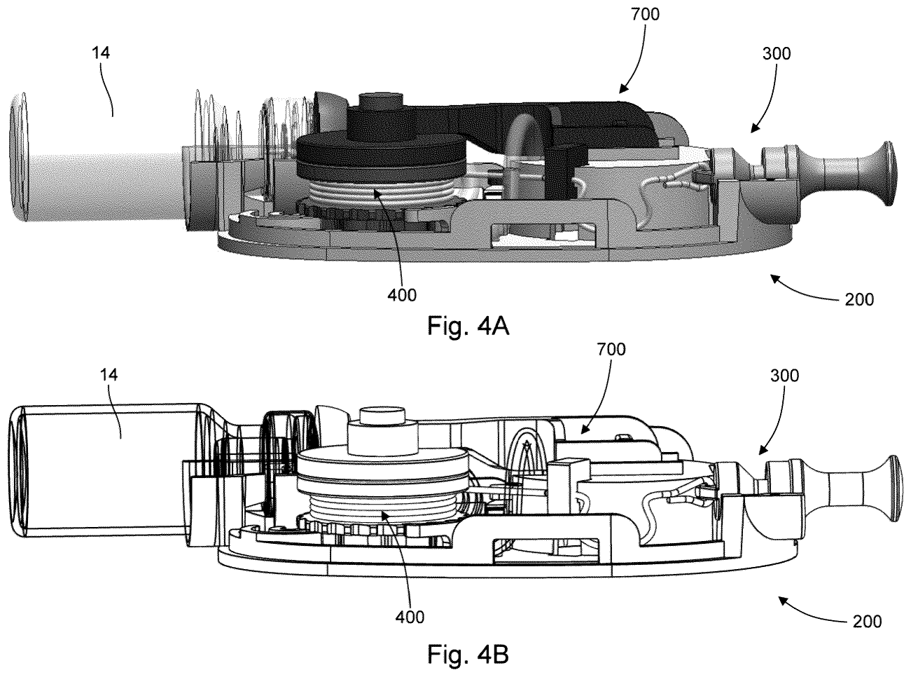

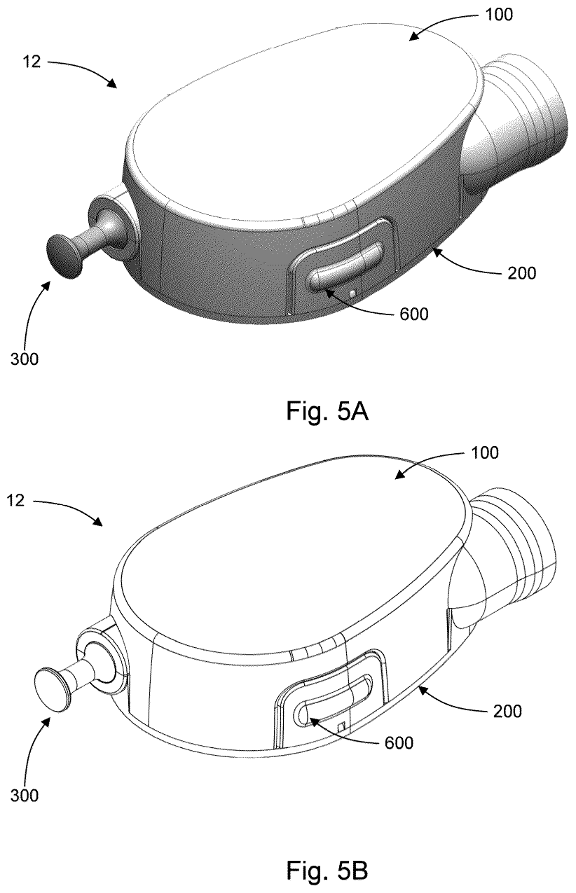

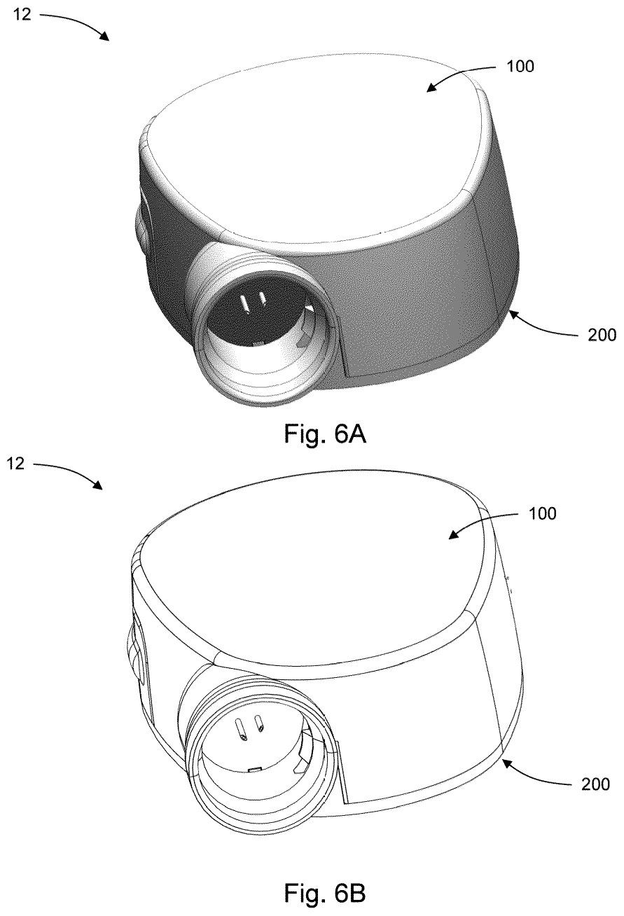

The middle support portion 422 includes an aperture 434 that extends longitudinally through the middle support portion 422. The aperture 434 is shaped and dimensioned to accept the cord 304 of the pull mechanism 300 which allows the cord 304 to detachably couple to the pump assembly 400. As depicted in FIGS. 4A and 4B, when coupled to the pump assembly 400 via the aperture 434, the cord 304 wraps around the middle support portion 422.

The ratchet 424 includes a top surface 424a and an opposed bottom surface 424b. Furthermore, the middle support portion 422 extends vertically between the bottom surface 428b of the base 428 and the top surface 424a of the ratchet 424. The ratchet 424 also includes a plurality of teeth 436 that extend circumferentially around the ratchet 424 and extends vertically between the top surface 424a and the bottom surface 424b. Each tooth 436 includes a vertical surface 438 and an angled surface 440. As depicted in FIGS. 3A and 3B, the latch 240 is disposed between two teeth 436. When the pump assembly 400 is rotated in the clockwise direction, the angled surfaces contact the latch 240 as the pump assembly is rotated. When the pump assembly 400 is rotated in the counterclockwise direction, a vertical surface 438 of a tooth 436 is pushed into the latch 240 which prevents the pump assembly 400 from rotating in the counterclockwise direction.

The cog 426 extends vertically from and perpendicular to the bottom surface 424b of the ratchet 424. The cog 426 includes a bottom surface 426a and the pump portion 404 includes a bottom cylinder 442 that extends vertically from and perpendicular to the surface 426a of the cog 426. When the pump assembly 400 is disposed within the cartridge 12, the bottom cylinder 442 extends into the retention aperture 238 of the base 200 which aids in coupling the pump assembly 400 to the base 200 and maintains the pump assembly 400 in a given position while allowing the pump assembly 400 to rotate. The cog 426 includes a plurality protrusions 444. As will be discussed in further detail herein, when the pump assembly 400 rotates, the cog 426 acts as a peristaltic pump as the protrusions force a pharmaceutical through the first tube 22 via positive displacement.

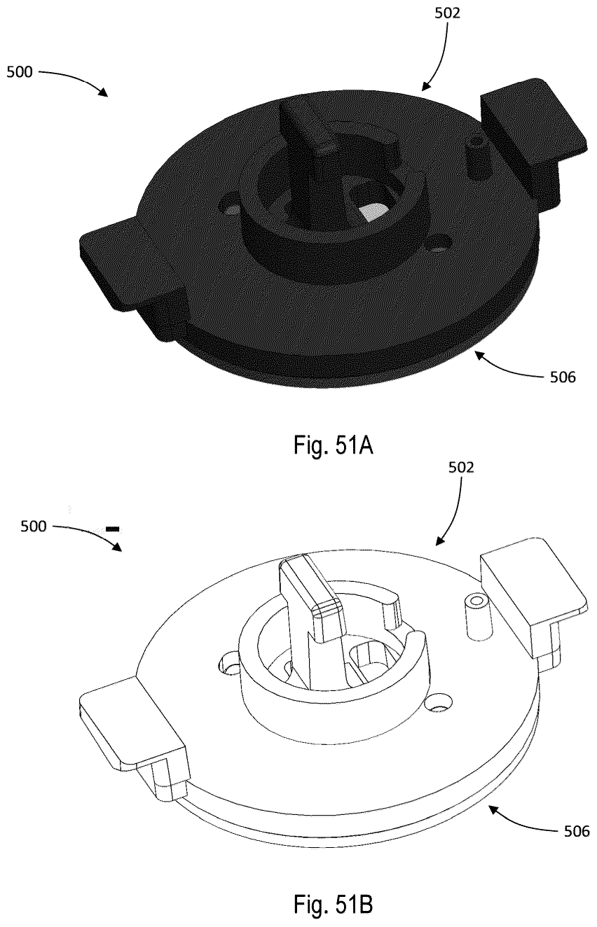

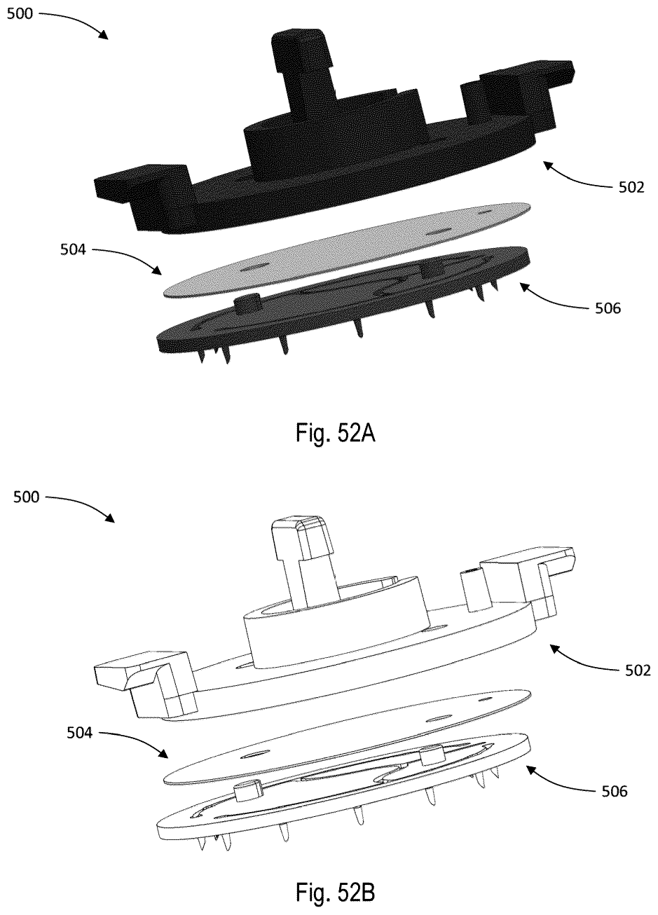

Referring now to FIGS. 51A, 51B and 52A and 52B, the microneedle array assembly 500 is shown in accordance with an exemplary embodiment. In this embodiment, the microneedle array assembly 500 includes a microneedle array holder 502, an adhesive film 504, and a microneedle array platform 506.

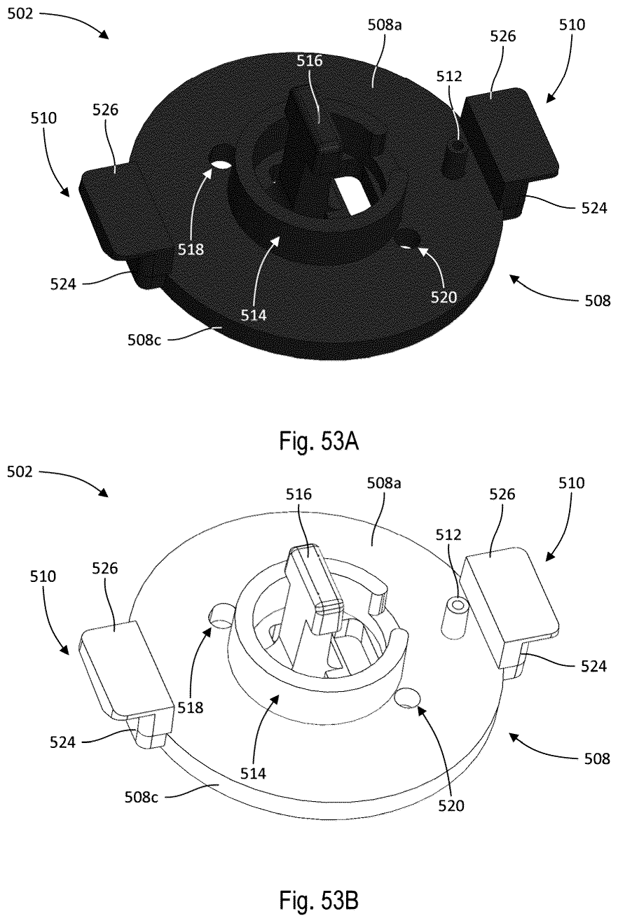

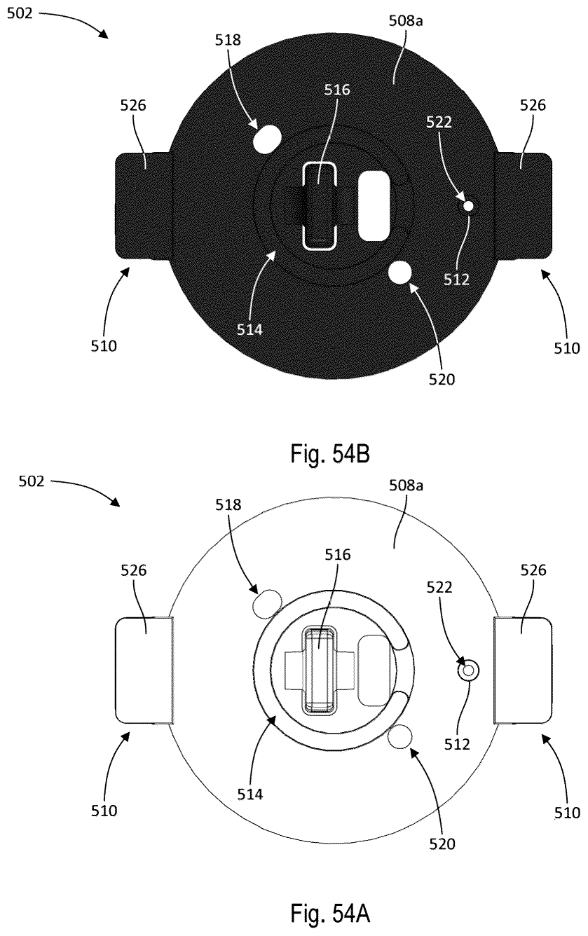

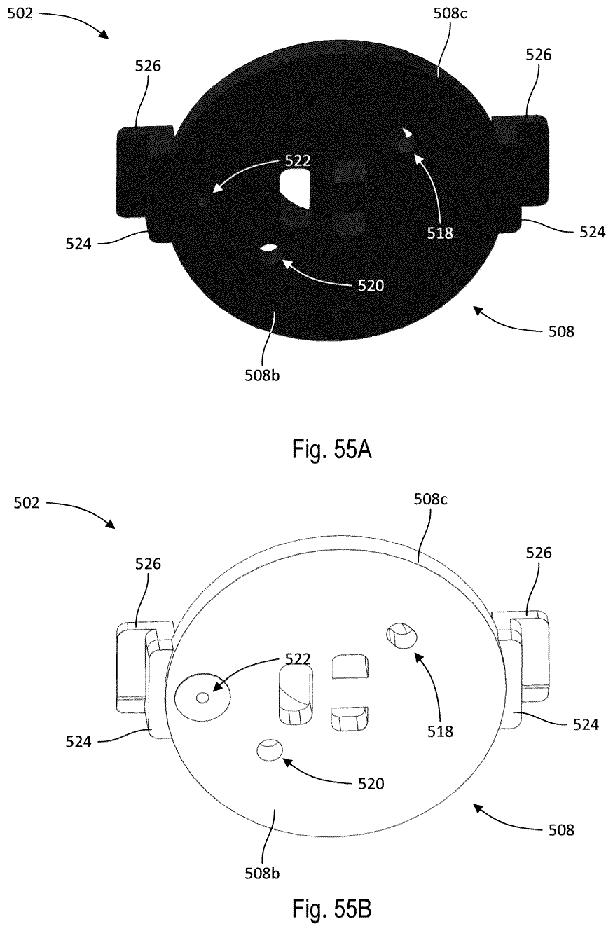

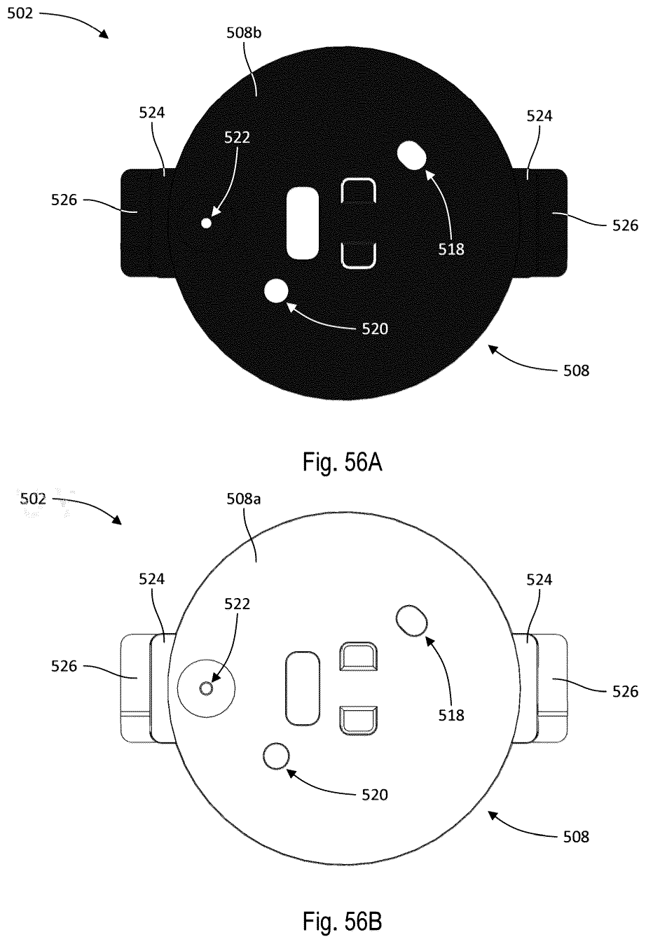

As depicted in FIGS. 53A and 53B-56A and 56B, the microneedle array holder 502 includes a circular base 508 extensions 510, a hollow cylinder 512, a circular projection 514, and a T-shaped column 516. The base 508 includes a top surface 508a, an opposed bottom surface 508b, and a side surface 508c that extends vertically between and perpendicular to the top surface 508a and the bottom surface 508b. The base 508 further includes a first circular opening 518 and a second circular opening 520 positioned on opposing sides of the circular projection 514. The base 508 also includes a circular aperture 522 that is positioned adjacent to an extension 510. The openings 518 and 520 and the circular aperture 522 extend through the base 508. That is the openings 518 and 520 and the circular aperture 522 extend between the top surface 508a and the bottom surface 508b of the base 508.

The extensions 510 include a vertical portion 524 and a horizontal portion 526. The vertical portion 524 extends longitudinally from and perpendicular to the side surface 508c of the base 508 and extends vertically from and perpendicular to the top surface 508a of the base 508. The horizontal portion 526 extends longitudinally from and perpendicular to the vertical portion 524.

The hollow cylinder 512 extends vertically from and perpendicular to the top surface 508a of the base 508. Furthermore, the hollow cylinder 512 extends circumferentially around the circular aperture 522 such that an opening extends through the hollow cylinder 512 and through the base 508.

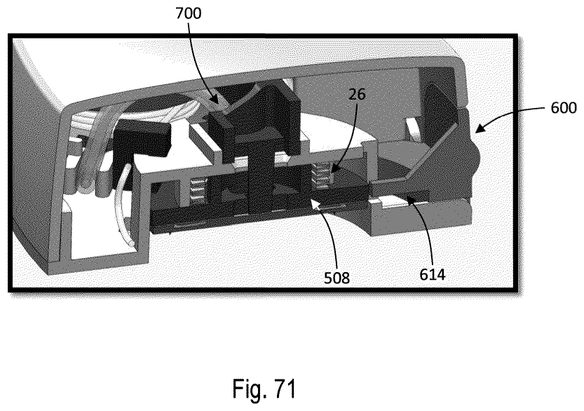

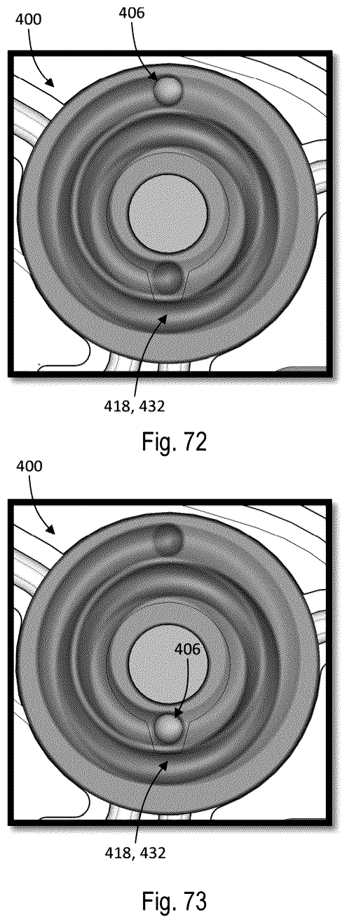

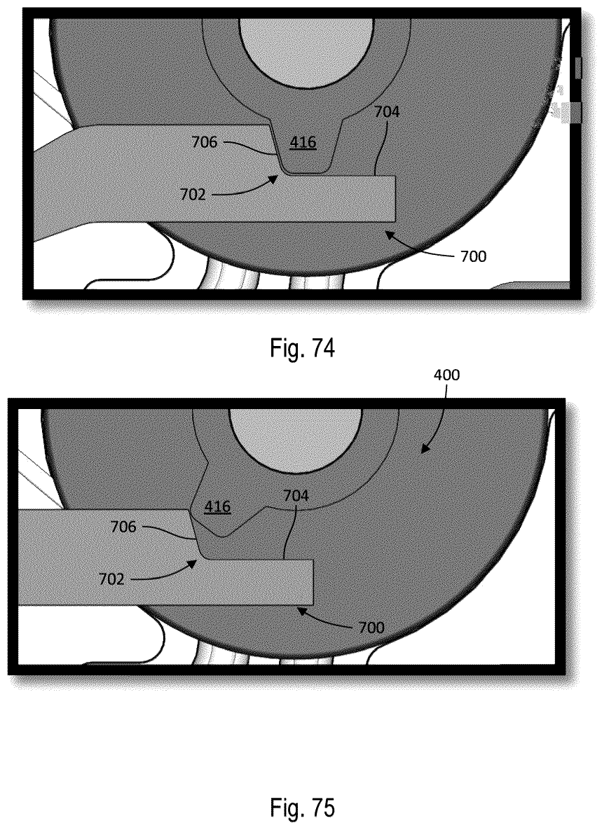

The circular projection 514 extends vertically from and perpendicular to the top surface 508a of the base 508 and surrounds the T-shaped column 516. As depicted in FIGS. 71, 76, and 77 the cartridge 12 includes an injection spring 26 that extends around the circular projection 514. As will be discussed in further detail herein, the injection spring 26 aids in moving the microneedle array assembly 500 from an undeployed position to a deployed position.

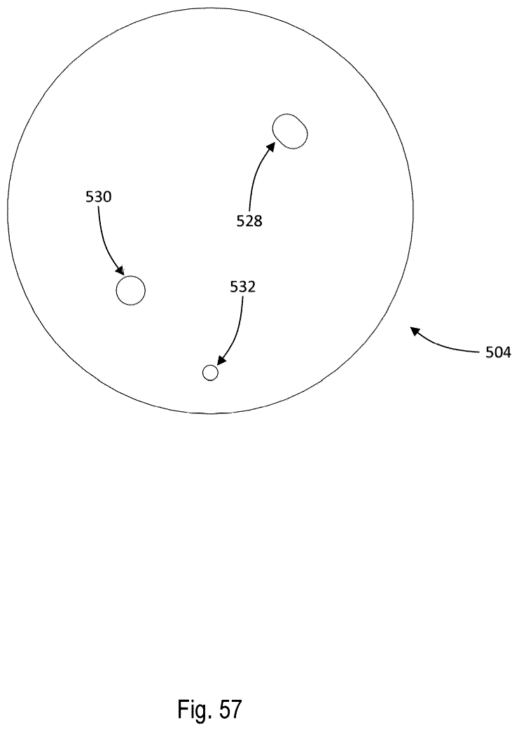

Referring now to FIG. 57, the adhesive film 504 is shown in accordance with an exemplary embodiment. The adhesive film 504 couples the microneedle array holder 502 to the microneedle array platform 506 and includes top surface and an opposed bottom surface. The top surface attaches to the microneedle array holder 502 and the bottom surface attaches to the microneedle array platform 506. The adhesive film 504 further includes a first opening 528, a second opening 530, and a third opening 532. When the adhesive film 504 is attached to the bottom surface of the base 508 of the microneedle array holder 502, the first opening 518 and the second opening 520 of the base 508 align with the first opening 528 and the second opening 530 of the film 504 respectively. Furthermore, when the adhesive film 504 is attached to the bottom surface of the base 508 of the microneedle array holder 502, the aperture 522 of the base 508 aligns with the third opening 532 of the film 504.

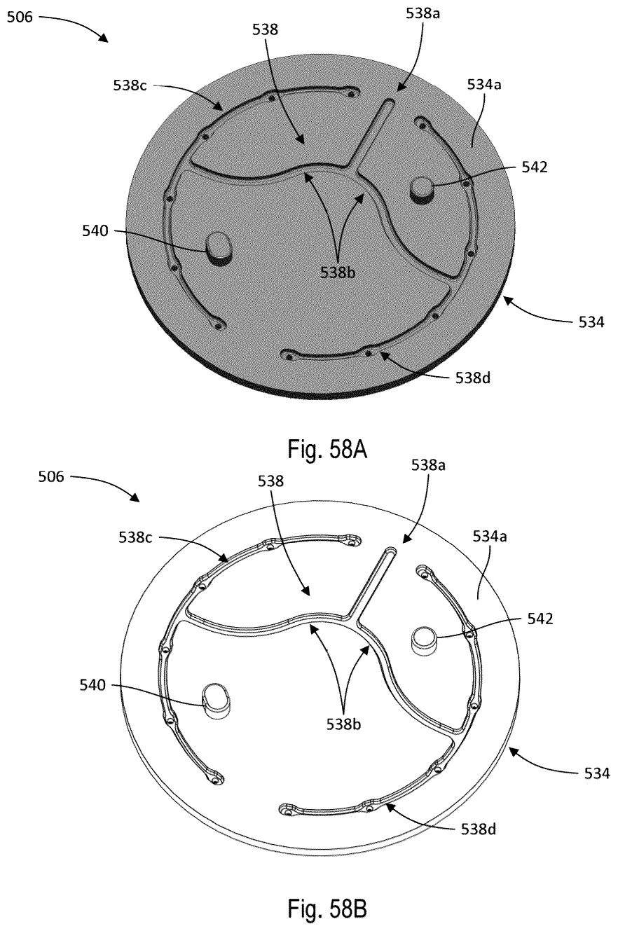

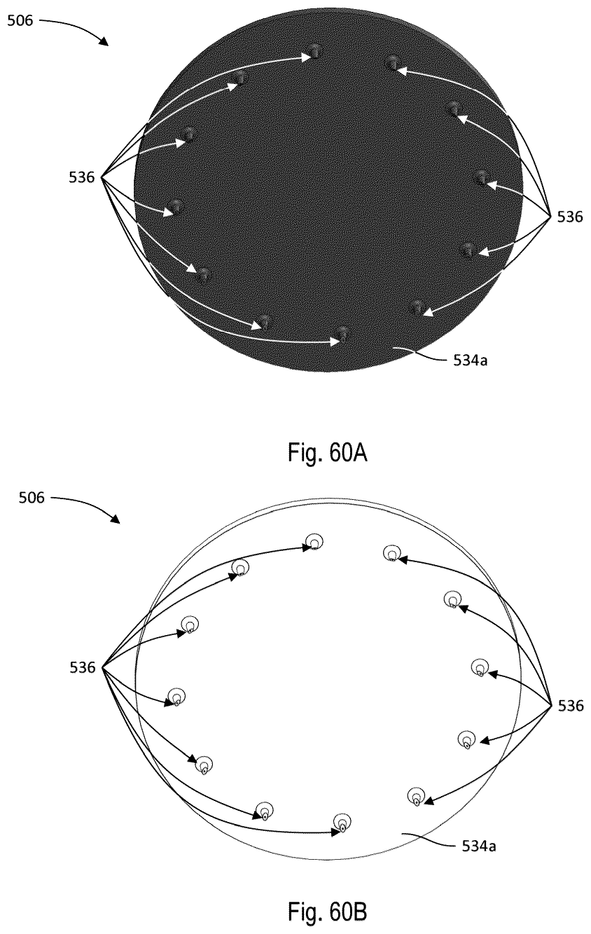

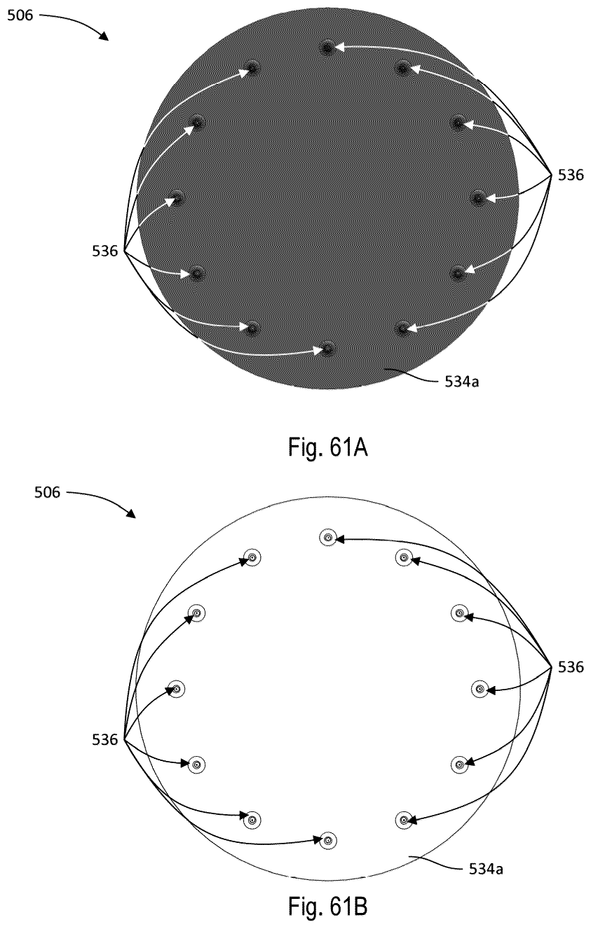

Referring now to FIGS. 58A and 58B-61A and 61B, the microneedle array platform 506 is shown in accordance with an exemplary embodiment. In this embodiment, the microneedle array platform 506 includes a base 534 that supports a plurality of hollow microneedles 536.

The base 534 of the microneedle array platform 506 includes a top surface 534a and an opposed bottom surface 534b. The plurality of microneedles 536 extend vertically from and perpendicular to the bottom surface 534b. The microneedles 536 are hollow such that the microneedles 536 include a lumen that extends through the base 534 such that an end of a microneedle 536 is in open communication with the top surface 534a of the base 534.

The top surface 534a of the base 534 defines an open fluidic channel 538 (e.g., a microfluidic channel) that includes a first portion 538a, a second portion 538b, a third portion 538c and a similar fourth portion 538d. The first portion 538a is generally linear, the second portion 538b extends in a T-junction from the first portion 538a and the third portion 538c and the fourth portion 538d extend from the second portion 538b. The third portion 538c and the fourth portion 538d extend around the circumference of the base 534. The openings of the lumens of the microneedles 536 extend to the third portion 538c and the fourth portion 538d of the open fluidic channel 538. As such, the lumens of the microneedles 536 are in open communication with the first portion 538a of the fluidic channel 538 via the second portion 538b, third portion 538c, and the fourth portion 538d.

The base 534 further includes a first extension 540 and a second extension 542 that extend vertically from and perpendicular to the top surface 534a of the base 534. When the top surface 534a of the base 534 is attached to the adhesive film 504, the first extension 540 extends through the first opening 528 and the second extension 542 extends through the second opening 530 of the adhesive film 504. Furthermore, when the microneedle array platform 506 is coupled to the microneedle array holder 502 via the adhesive film 504 the first extension 540 extends through the first circular opening 518 and the second circular opening 520 of the base 534 of the microneedle array platform 506 respectively. In another embodiment, the adhesive film 504 may be omitted and, in such an embodiment, the microneedle array holder 502 may be coupled to the microneedle array platform 506 via ultrasonic welding.

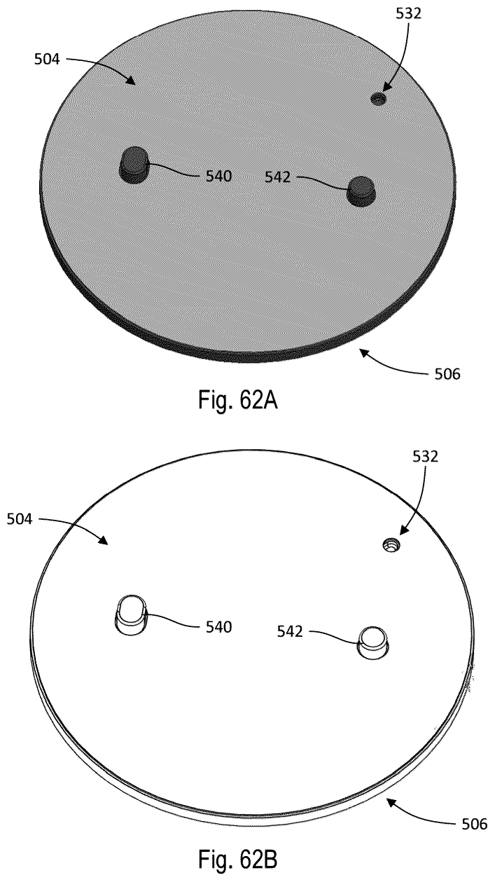

As depicted in FIGS. 62A and 62B, the adhesive film 504 covers the top surface 534a of the base 534. In this position, the opening 532 is disposed vertically above a segment of the first portion 538a of the fluidic channel 538 which seals all but this portion of the fluidic channel 538. When the microneedle array assembly 500 is assembled, the opening 532 of the adhesive film 504 (and therefore a portion of the first portion 538a of the fluidic channel 538) is positioned vertically below the circular aperture 522. As such, the fluidic channel 538 is in open communication with the aperture 522. As further depicted in FIG. 34A, 34B, 35A, and 35B, when the cartridge 12 is assembled, the first tube 22 is connected to the hollow cylinder 512 and as such, the first tube 22 is in open communication with the fluidic channel 538 via the hollow cylinder 512.

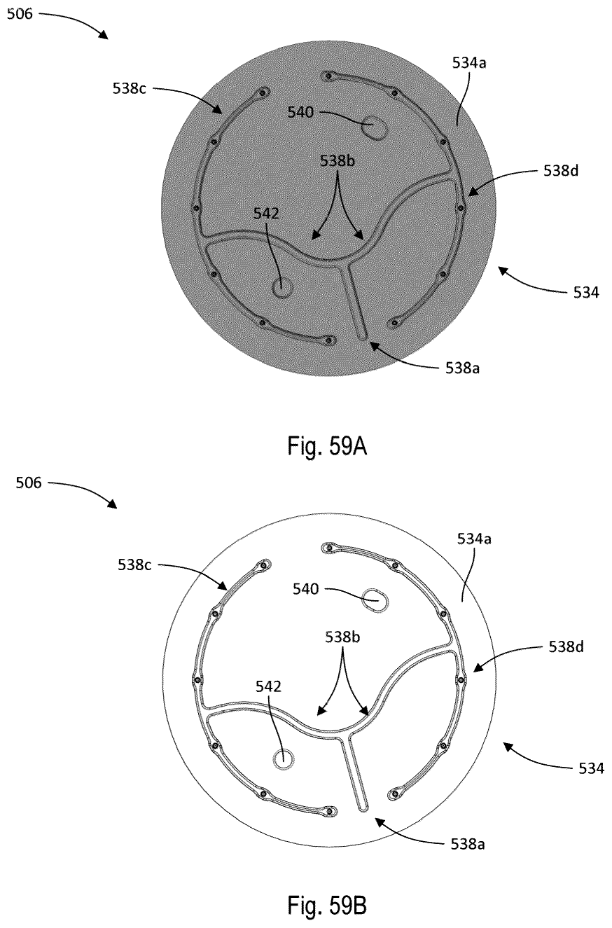

As will be discussed in further detail herein, the dermal patch system 10 delivers the pharmaceutical to a subject via the microneedles 536. As depicted in FIGS. 59A and 59B, an equal number of microneedles 536 are disposed on opposite sides of an end of the second portion 538b. That is, as depicted in FIGS. 59A and 59B, three microneedles are disposed on one side of the second portion 538b and three microneedles 536 are disposed on the opposite side of the second portion 538b of the fluidic channel 538. Providing the same number of microneedles 536 on opposite ends of the second portion 538b of the fluidic channel 538 allows substantially the same amount of pharmaceutical to flow through each microneedle 536 at about a same flow rate (e.g., about 130 μl/min).

While the dermal patch system 10 is depicted as including twelve microneedles 536, in other embodiments, the dermal patch system may include more or less microneedles 536 each in communication with the fluidic channel 538. Furthermore, the microneedles 536 may have the same or varied length. For example, the length of the microneedles may be in a range of about 1 mm-about 3 mm. The length of the microneedles 536 determines a depth into the dermis of the subject that the pharmaceutical is delivered. Furthermore, subjects may have varying depths of dermal layers. For example, the epidermis of one subject may be thicker or thinner compared to the epidermis of another subject. Providing a dermal patch system 10 with microneedles 536 of varying lengths may ensure that a pharmaceutical is delivered to the dermis of a subject as at least one of the microneedles 536 may extend through the epidermis of the subject. In some embodiments, the lengths of the microneedles 536 may be varied by a factor in a range of at least 10% to about 20%. In one embodiment, wherein the pharmaceutical is administered intradermally, the microneedles 536 may have a length between about 1 mm and about 2 mm. In another embodiment, wherein the pharmaceutical is administered subcutaneously, the microneedles 536 may have a length between about 2 mm and about 3 mm.

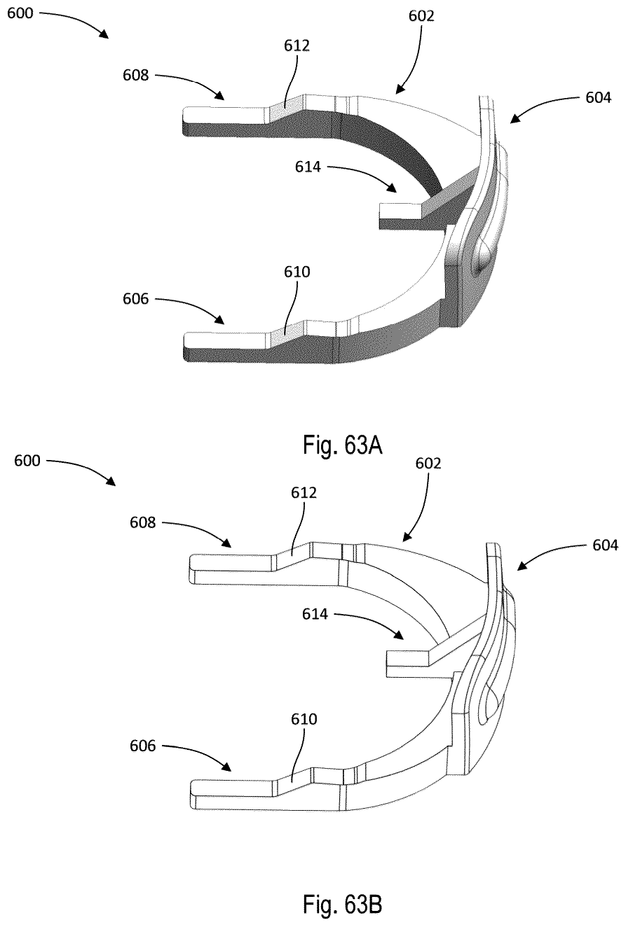

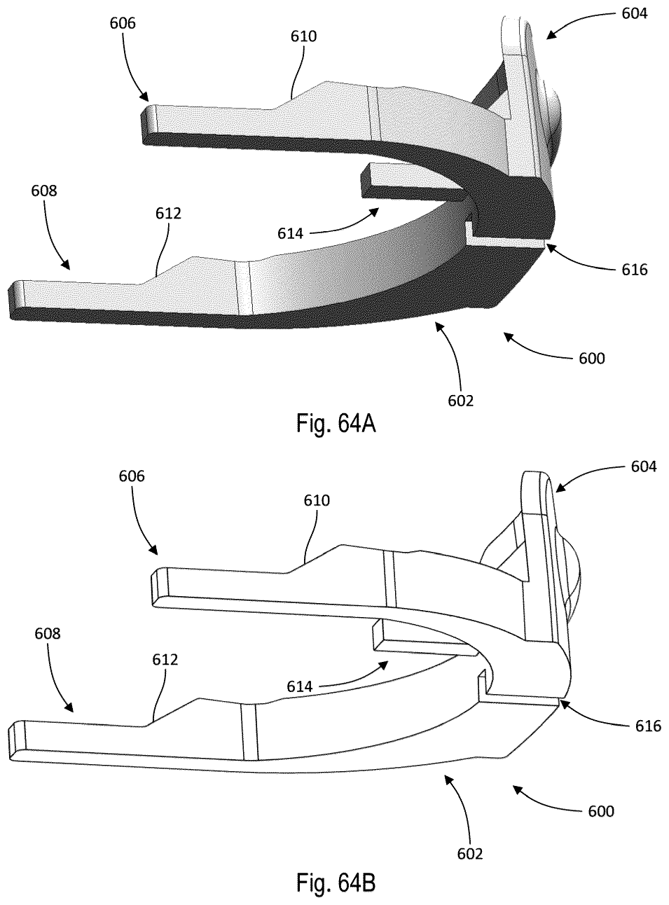

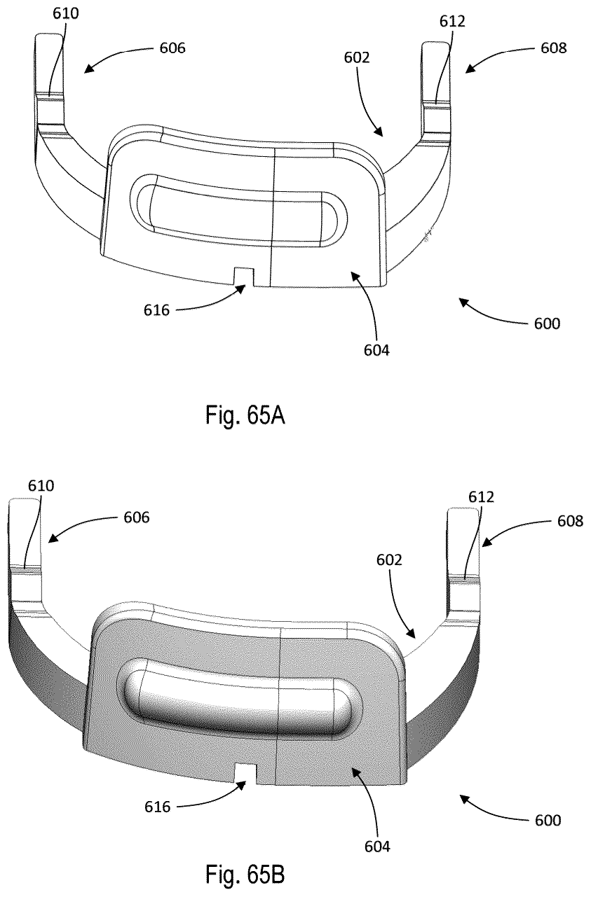

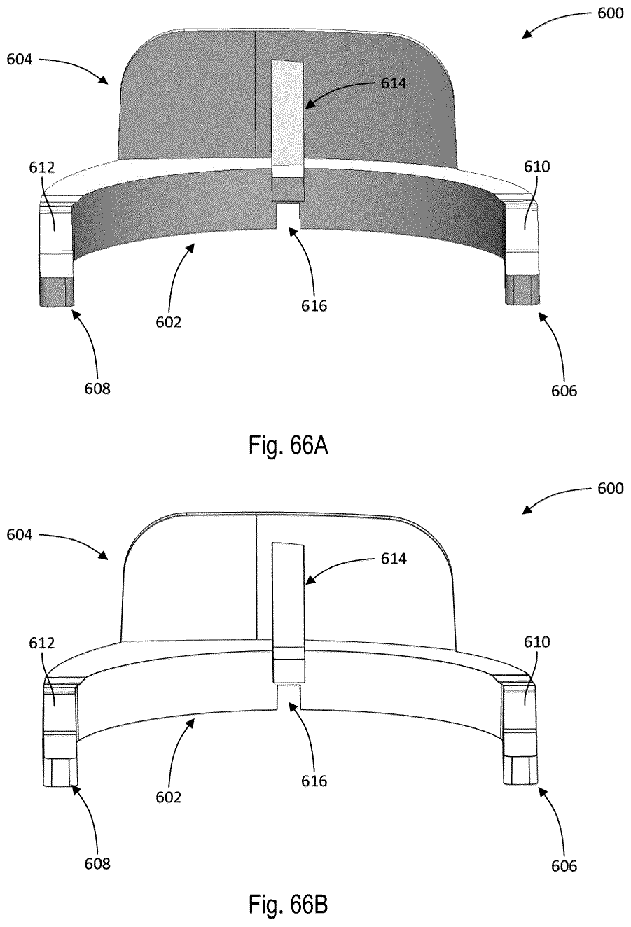

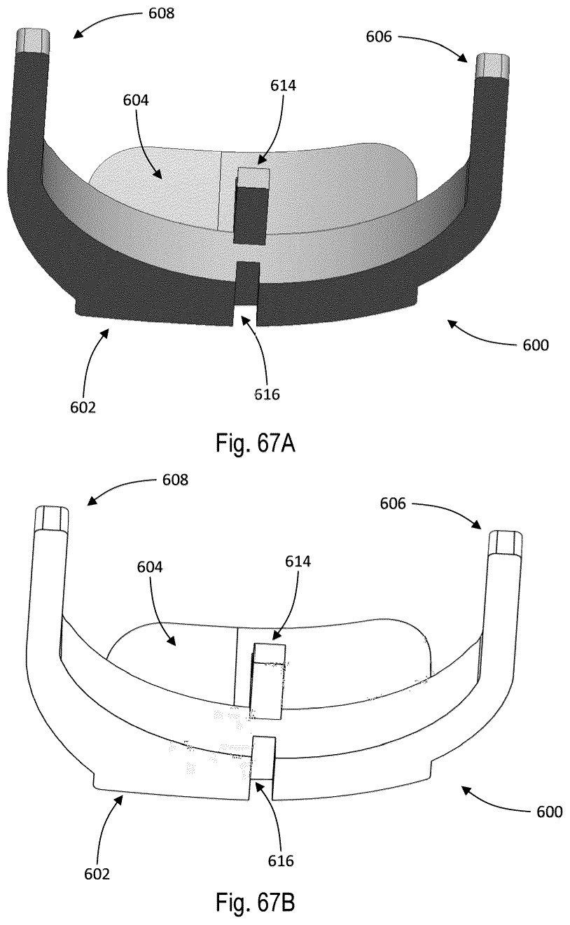

Referring now to FIGS. 63A and 63B-67A and 67B, the retraction button 600 is shown in accordance with an exemplary embodiment. The retraction button 600 includes a U-shaped base 602 and a vertical button portion 604 that extends vertically from and perpendicular to the U-shaped base 602. The U-shaped base 602 includes a first arm 606 and a second arm 608. The first arm 606 and the second arm 608 include an angled surface 610 and 612 respectively. The U-shaped base 602 has a shape that is similar to the shape of the microneedle array housing 244 such that the first arm 606 and the second arm 608 extend around microneedle array housing 244. The retraction button 600 further includes a blocking feature 614. The blocking feature 614 extends vertically from and perpendicular to the U-shaped base 602 and extends longitudinally from and perpendicular to the vertical button portion 604.

The retraction button 600 further includes a gap 616 that extends through the U-shaped base 602 and the vertical button portion 604. The gap 616 is shaped and dimensioned to extend over the third button guide 274.

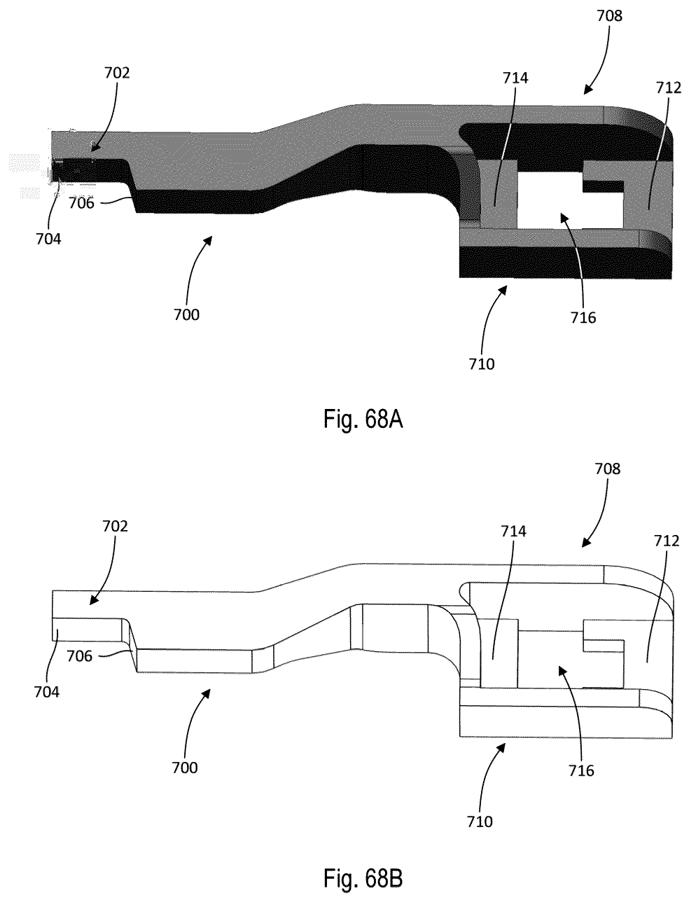

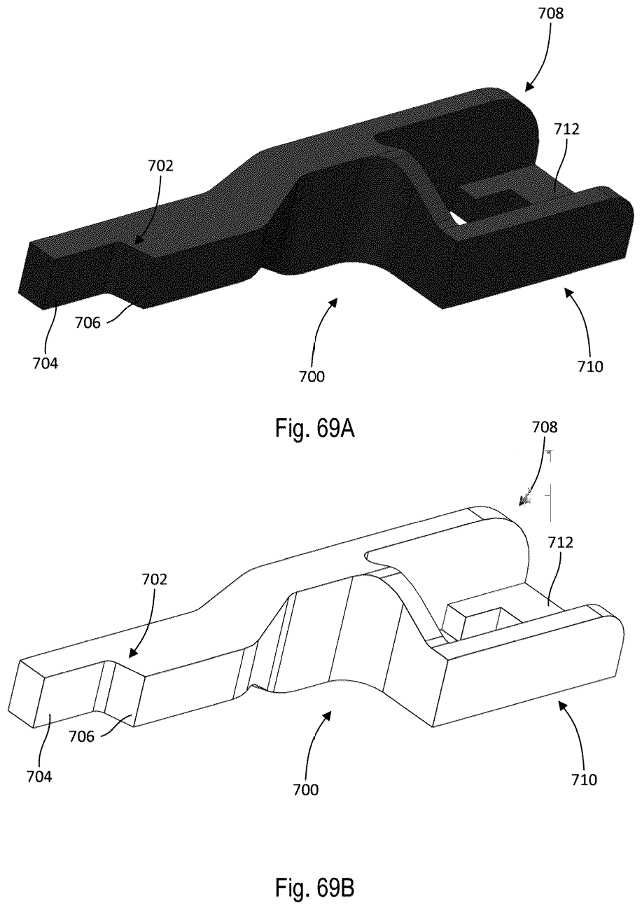

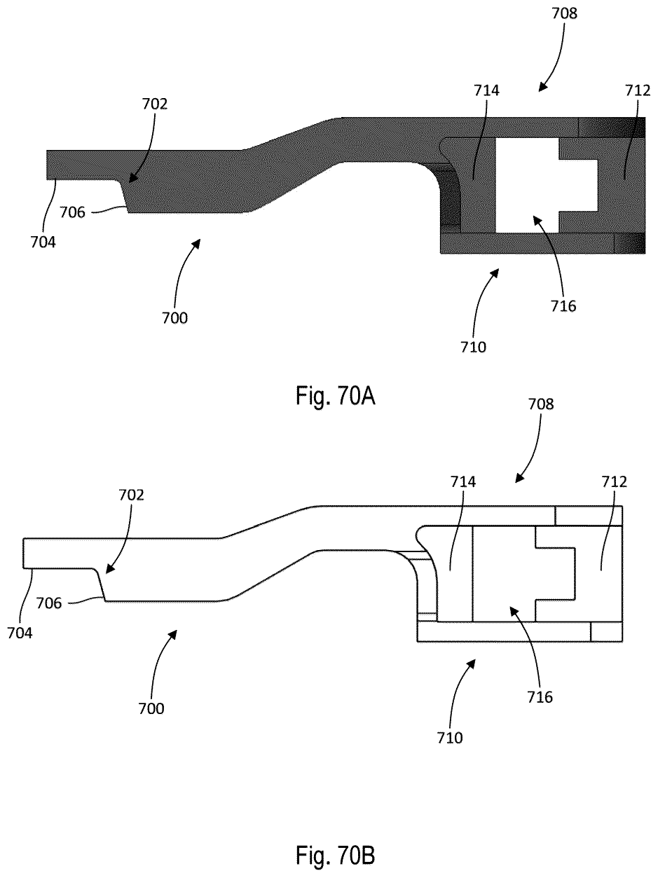

As depicted in FIGS. 3A and 3B, the cartridge 12 further includes a trigger 700. With particular reference to FIGS. 68A and 68B-70A and 70B, the trigger 700 includes a notch 702 that is defined by a first surface 704 and a second surface 706. The trigger 700 further includes a first outer wall 708 and a second outer wall 710 that is parallel to the first outer wall 708. The trigger 700 also includes a first bottom wall 712 and a second bottom wall 714 that extend between the first outer wall 708 and the second outer wall 710. The first bottom wall 712 and the second bottom wall 714 define a T-shaped opening 716 that also extends between the first outer wall 708 and the second outer wall 710.

When the trigger 700 is inserted into the cartridge 12, a portion of the bottom surface of the trigger 700 rests upon the outer surface 248a of the microneedle array housing 244 and another portion of the bottom surface of the trigger 700 rests upon the top surface 408a of the trigger portion 402 of the pump assembly 400. Furthermore, the first outer wall 708 and the second outer wall 710 are disposed between the first and second trigger guides 262.

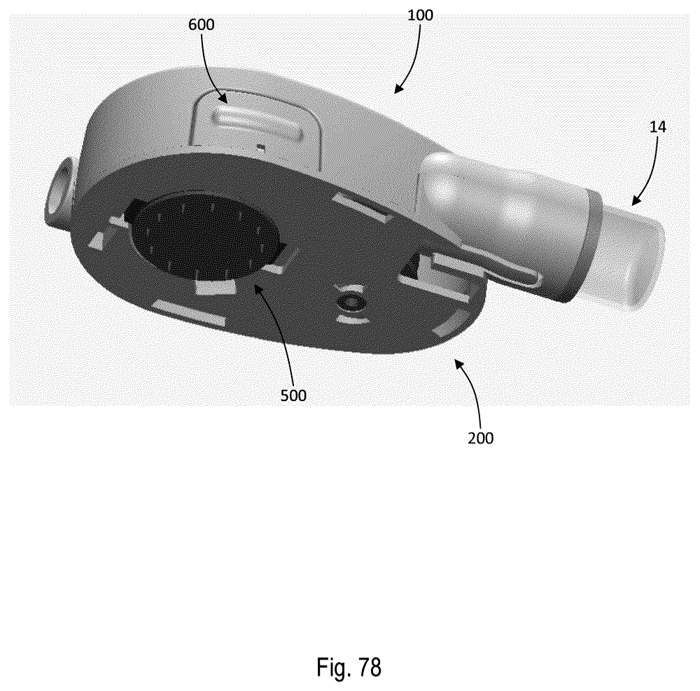

The cartridge 12 is moveable between an undeployed position (FIGS. 3A and 3B) to a deployed position (FIGS. 77 and 78).

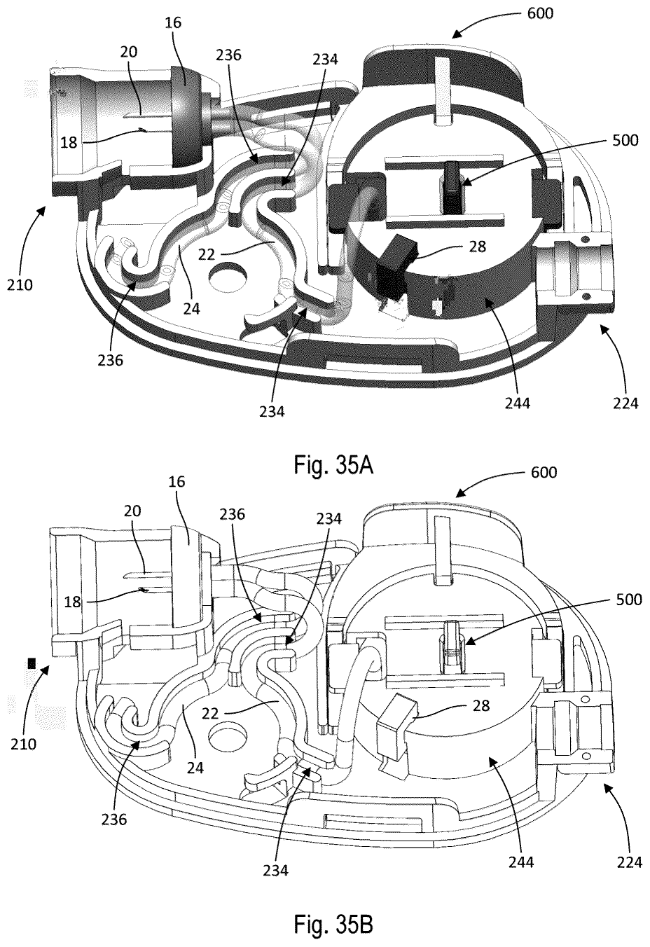

In the undeployed position, the handle 302 of the pull mechanism 300 is disposed within the handle receptacle of the cartridge 12 and the cord 304 of the pump assembly 400 extends through the aperture 434 and wraps around the middle support portion 422 of the pump assembly 400. As depicted in FIGS. 34A, 34B, 35A, and 35B, the cartridge 12 includes a tensioner 28. The tensioner 28 extends through the first tensioner opening 256 and the second tensioner opening 258 of the microneedle array housing 244 which allows the tensioner 28 to couple to the microneedle array housing 244. Furthermore, the tensioner 28 includes an aperture that allows the cord 304 to extend therethrough. As further depicted in Figs, 3A and 3B, when the cartridge 12 is in the undeployed position, the tensioner 28 causes the portion of the cord 304 that wraps around middle support portion 422 of the pump assembly 400 to be taut while portion of the cord 304 that is between the handle 302 and the tensioner 28 has some slack.

As further depicted in FIG. 74, in the undeployed position, the extension 416 of the pump assembly 400 is disposed within the notch 702 such that the extension 416 contacts the first surface 704 and the second surface 706 of the notch 702.

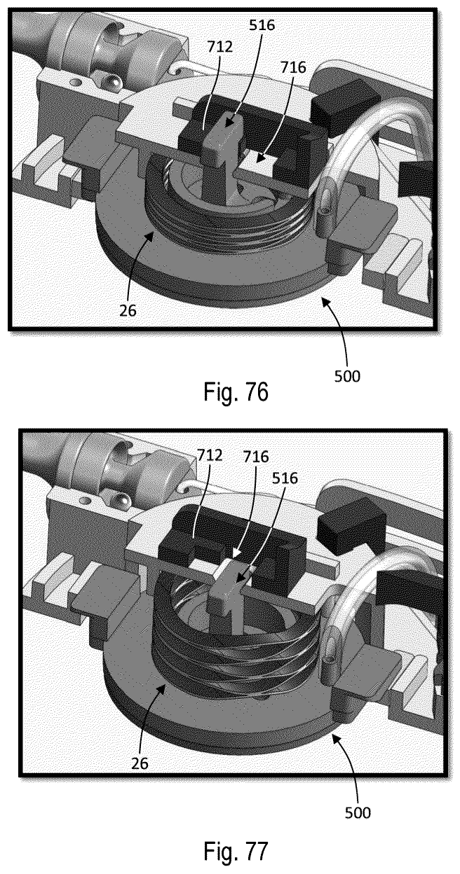

Furthermore, in the undeployed position, the T-shaped column 516 of the microneedle array holder 502 extends through the aperture 260 of the microneedle array housing 244. In this position, the T-shaped column 516 also extends through the T-shaped opening 716 and rests upon the first bottom wall 712 which prevents microneedle array assembly 500 from moving through the microneedle array aperture 242 of the base 200. As such, the microneedles 536 are retained within the cartridge 12 when the microneedle array assembly 500 is in the undeployed position. Also, as depicted in FIG. 76, the microneedle array assembly 500 compresses the injection spring 26 between the top surface 508a of the microneedle array assembly 500 and the inner surface 248b of the microneedle array housing 244, such that the spring is in a compressed state.

As depicted in FIGS. 34A, 34B, 35A, and 35B, when the retraction button 600 is in the undeployed position, the first arm 606 of the retraction button 600 is disposed between the first button guide 270 and the outer surface 246a of the microneedle array housing 244 and the second arm 608 of the retraction button 600 is disposed between the second button guide 272 and the outer surface 246a of the microneedle array housing 244. Furthermore, in this position, the first and second arms 606 and 608 are positioned below the extensions 510 of the microneedle array holder 502 and the third button guide 274 extends through the gap 616. When the microneedle array assembly 500 is in the undeployed position, the microneedle array holder 502 prevents a user from activating the retraction button 600 by pushing it into the cartridge (also referred to moving the retraction button 600 to a deployed position from an undeployed position) as the blocking feature 614 extends through the opening 254 of the microneedle array housing 244 and contacts base 508 which prevents the retraction button 600 from moving.

In order to move the cartridge to the deployed position, the cartridge 12 must be placed in a substantially vertical position. As depicted in FIGS. 3A and 3B, when the cartridge 12 is not in a substantially vertical position, the ball 21 is disposed within the ball retention groove. When the ball 21 is within the ball retention groove and the handle 302 is pulled, the angled surface 316 of the handle 302 contacts the ball 21 and pushes the ball 21 into the inner surface of the handle receptacle which prevents the handle 302 from being pulled further. When the cartridge 12 is in a substantially vertical position, the ball 21 falls into and is retained by the rounded inner surface 312 of the handle 302 which allows a user to pull the handle 302 to remove the pull mechanism 300 from the cartridge 12.

Pulling the pull mechanism 300 causes the cartridge 12 to move from the undeployed position to the deployed position. More particularly, pull mechanism 300 causes the pump portion 404 to rotate in a clockwise direction. As the pump portion 404 rotates, the latch 240 engages with various teeth 436 of the pump portion 404. This engagement may slow a rotation of the pump portion 404 as the engagement must be overcome to cause the pump portion 404 to rotate. In addition, the engagement provides a tactile feel to the user and stabilizes a rate of pull for the user. When the pump portion 404 rotates, the protrusions 444 of the cog 426 compress the first tube 22 and the second tube 24 at various locations as the cog 426 rotates. When the vial 14 is inserted into the cartridge 12 this compression causes the cog 426 to act as a peristaltic pump by causing an amount of a pharmaceutical to travel through the first tube 22 and pumps air into the vial 14 via the second tube 24 which aids in expelling pharmaceutical from the vial 14.

Furthermore, when the pump portion 404 rotates, the ball 406 travels within the spiral ball retention groove until the ball 406 reaches an inner end of the spiral ball retention groove. While the ball 406 is traveling, the trigger portion 402 remains stationary. When the ball 406 reaches the end of the spiral ball retention groove, the ball engages the end of the spiral groove 418 of the trigger portion 402 and engages with the end of the spiral groove 432 of the pump portion 404 which causes the trigger portion 402 to rotate with the pump portion 404. As will be discussed in further detail herein, the rotation of the trigger portion 402 causes the microneedle array assembly 500 to move to the deployed position for administering the pharmaceutical. As such, the pump portion 404 rotates (and as such pumps an amount of pharmaceutical through first tube 22) while the trigger position 402 remains stationary thereby priming an amount of pharmaceutical at the microneedles 536 before the microneedles 536 are deployed to puncture the subject's skin.

The amount the pump portion 404 rotates while the trigger portion 402 is stationary determines and can be proportional in various embodiments to an amount of pharmaceutical primed for administration. By way of example, in some embodiments, the amount of the pharmaceutical that is released from the vial for priming the microneedles can be proportional to a degree of rotation of the pump portion 404. That is, the more or less the pump portion 404 rotates while the trigger portion 402 is stationary, the more or less pharmaceutical is primed. As such, the amount the pump portion rotates before the ball 406 engages with the ends of the spiral grooves 418 and 432 may be increased or decreased by increasing or decreasing a distance covered by the grooves 418 and 432. In the embodiments disclosed herein, the pump portion 404 rotates three times before the ball 406 causes the trigger portion 402 to rotate with the pump portion 404. It is understood that this number of rotations may be increased or decreased by increasing or decreasing the length of the grooves 418 and 432 as needed.

When the ball 406 engages the ends of the grooves 418 and 432, the pump portion rotates in a clockwise direction such that the extension 416 rotates into the second surface 706 of the notch 702 of the trigger 700. This rotation pushes the trigger 700 from a first position away from the pump assembly 400 in the direction of arrow A to a second position. This movement causes the first bottom wall to move away from and disengage from the first bottom wall 712 of the trigger 700. When disengaged, the injection spring 26 is allowed to decompress and move the microneedle array assembly 500 to the deployed position. In this position, the microneedles 536 extend through the microneedle array assembly aperture 260 and can puncture the subject's skin when the cartridge 12 is affixed to the skin of the subject.

The pump assembly 400 continues to rotate until the end of the cord 304 releases from the pump assembly 400. That is, the pump assembly 400 continues to rotate until the end of the cord 304 passes through the aperture 434. As such, an amount of pharmaceutical pumped out of the vial 14 is proportional to the length of the cord 304 as a longer cord 304 allows for more rotation of the pump assembly 400 and a shorter cord 304 results in less rotation of the pump assembly 400 pump assembly 400.

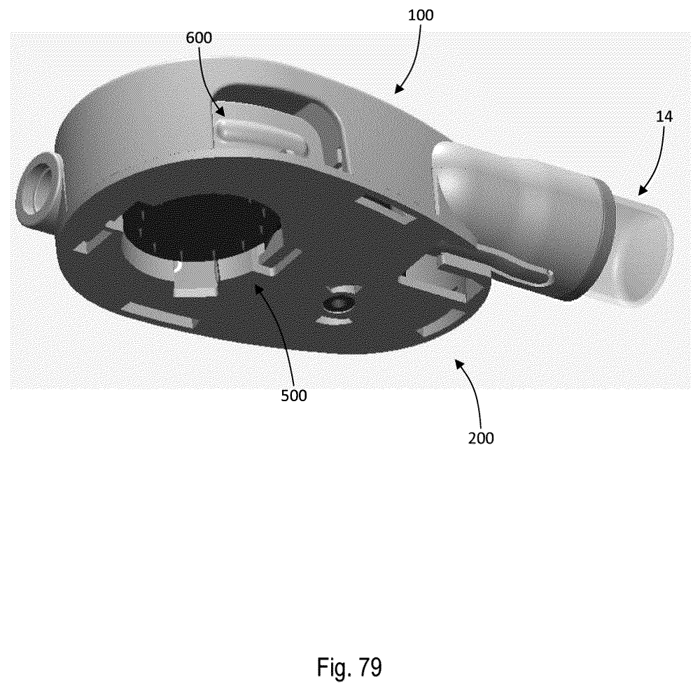

The microneedle array assembly 500 is moveable from the deployed position (FIG. 78) to a retracted position (FIG. 79).

When the microneedle assembly 500 is in the deployed position, the blocking feature 614 of the retraction button 600 no longer contacts the base 508 which allows a user to push the retraction button in the direction of arrow C and into the cartridge 12. When pushed, the first button guide 270, the second button guide 272 and the third button guide 274 direct the retraction button 600 towards the microneedle array housing 244. As the first arm 606 and the second arm 608 move, the angled surfaces 610 and 612 contact the extensions 510 which causes the microneedle array assembly 500 to move upward in the direction of arrow D and out of the subject's skin thereby compressing the injection spring 26.

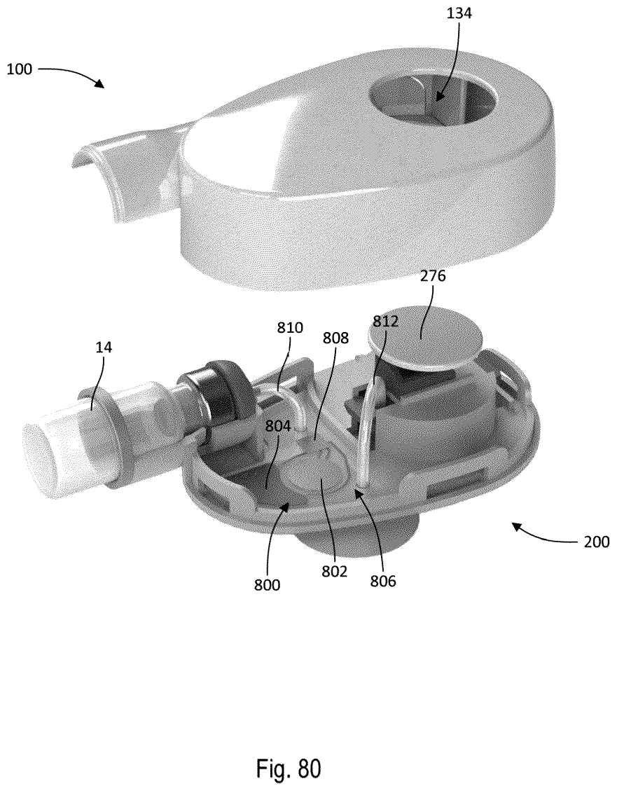

Referring now to FIG. 80, in another embodiment, the pump assembly 400 may be removed and replaced with a piezoelectric pump system 800. In such an embodiment, the piezoelectric pump system 800 can include a piezoelectric pump 802, a battery (e.g., a printed battery) 804, one or more laminate layers 806 stacked upon one another, a pump controller 808, a first tube 810 and a second tube 812. Furthermore, in such an embodiment, the stop 16 includes one hollow needle (e.g., the hollow needle 18) that is employed to connect the first tube 810 to the vial 14. The first tube 810 and the second tube 812 are connected to the piezoelectric pump 802 via one or more fluidic channels (e.g., microfluidic channels) (not shown). The second tube 812 is connected to the hollow cylinder 512 which allows the second tube 812 to carry a pharmaceutical to the microneedles 536.

In this embodiment, the cover 100 is modified to include a button aperture 134 that extends to the top wall 102. The base 200 includes a button 276. When the cover 100 is connected to the base 200 the button 276 extends through the button aperture 134. The button 276 is connected to the trigger 700 and when pushed, moves the trigger 700 from the first position to the second position to release the microneedle array assembly 500 as previously discussed herein. Furthermore, pushing the button 276 activates the piezoelectric pump 802 via a switch to pump the pharmaceutical from the vial 14 to the microneedles 536. After the pharmaceutical is administered, a user can push the retraction button 600 to retract the microneedles 536 into the

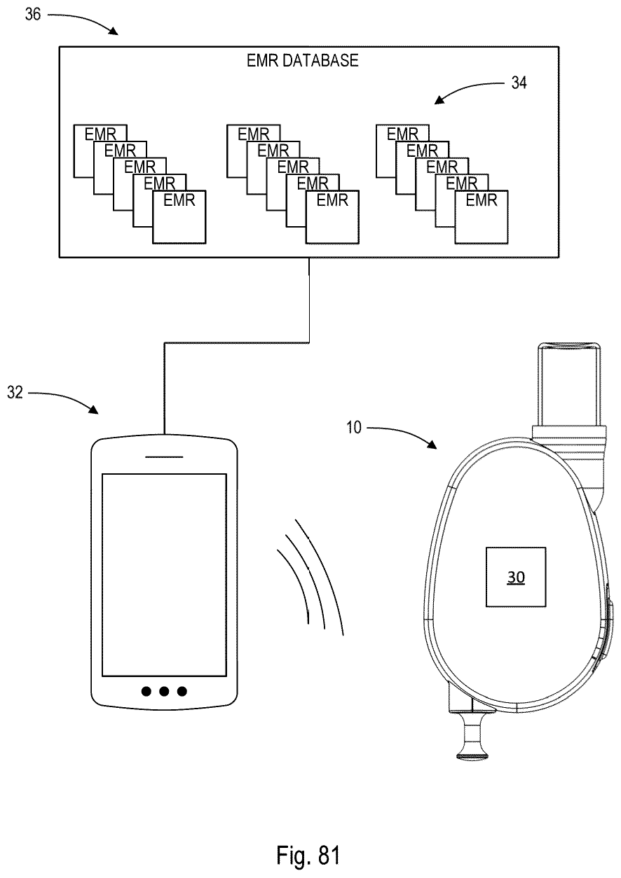

Referring now to FIG. 81, in some embodiments, the cover 100 includes a quick response (“QR”) code 30 disposed on the outer surface 102a of the top wall 102. As will be discussed in further detail herein, the QR code 30 can be associated with an electronic medical record (“EMR”) stored in an electronic medical record database.

In these embodiments, a user of a computer system 32 may scan the QR code 30 to view and/or update an EMR 34 that is associated with the QR code 30. The EMR 34 can be stored within an EMR database 36 that is in communication with the computer system 32. Furthermore, the QR code 30 may be employed to preserve the chain of custody of the dermal patch system 10.

The computer system 32 may include an application that provides access to the EMR database 36 via a network connection and allows a user to photograph or scan the QR code 30. As shown in FIG. 81 the EMR database 36 includes a plurality of EMRs 34 each of which is associated with an individual subject. The application causes the computer system 32 to scan or retrieve an image of the QR code 30, analyze the QR code 30 and associate the QR code 30 with an EMR 34. In some embodiments, the computer system 32 may then update the associated EMR 34 to indicate the subject has been administered the pharmaceutical from the vial 14. The computer system 32 may update the EMR 34 automatically or based on a user input.

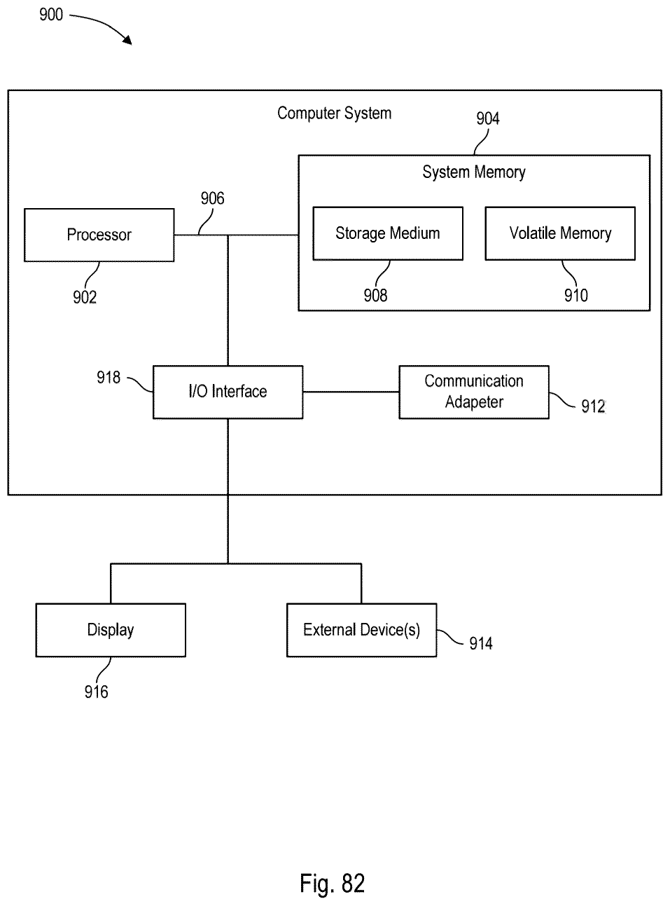

Referring now to FIG. 82, a computer system 900 is shown in accordance with an exemplary embodiment. The computer system 900 may serve as any computer system disclosed herein (e.g., the computer system 32). As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 82, the computer system 900 includes one or more processors or processing units 902, a system memory 904, and a bus 906 that couples the various components of the computer system 900 including the system memory 904 to the processor 902. The system memory 904 includes a computer readable storage medium 908 and volatile memory 910 (e.g., Random Access Memory, cache, etc.). As used herein, a computer readable storage medium includes any media that is capable of storing computer readable; program instructions and is accessible by a processor. The computer readable storage medium 908 includes non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 906 may be one or more of any type of bus structure capable of transmitting data between components of the computer system 900 (e.g., a memory bus, a memory controller, a peripheral bus, an accelerated graphics port, etc.).

The computer system 900 may further include a communication adapter 912 which allows the computer system 900 to communicate with one or more other computer systems/devices via one or more communication protocols (e.g., Wi-Fi, BTLE, etc.) and in some embodiments may allow the computer system 900 to communicate with one or more other computer systems/devices over one or more networks (e.g., a local area network (LAN), a wide area network (WAN), a public network (the Internet), etc.).

In some embodiments, the computer system 900 may be connected to one or more external devices 914 and a display 916. As used herein, an external device includes any device that allows a user to interact with a computer system (e.g., mouse, keyboard, touch screen, etc.). An external device 914 and the display 916 may be in communication with the processor 902 and the system memory 904 via an Input/Output (I/O) interface 918.

The display 916 may display a graphical user interface (GUI) that may include a plurality of selectable icons and/or editable fields. A user may use an external device 914 (e.g., a mouse) to select one or more icons and/or edit one or more editable fields. Selecting an icon and/or editing a field may cause the processor 902 to execute computer readable program instructions stored in the computer readable storage medium 908. In one example, a user may use an external device 914 to interact with the computer system 900 and cause the processor 902 to execute computer readable program instructions relating to at least a portion of the steps of the methods disclosed herein.

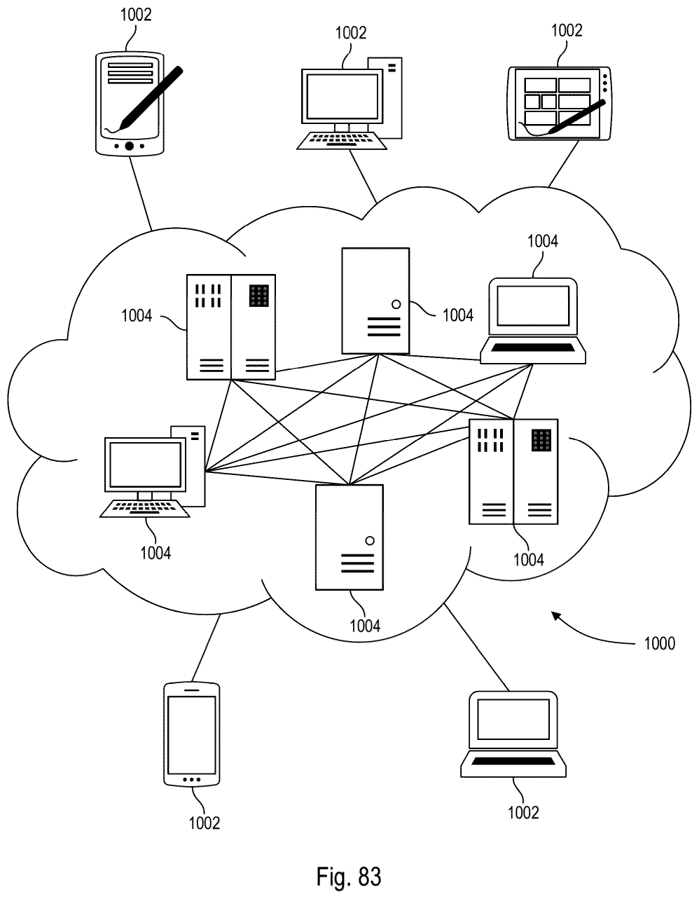

Referring now to FIG. 83, a cloud computing environment 1000 is depicted in accordance with an exemplary embodiment. The cloud computing environment 1000 is connected to one or more user computer systems 1002 and provides access to shared computer resources (e.g., storage, memory, applications, virtual machines, etc.) to the user computer systems 1002. As depicted in FIG. 83, the cloud computing environment includes one or more interconnected nodes 1004. Each node 1004 may be a computer system or device local processing and storage capabilities. The nodes 1004 may be grouped and in communication with one another via one or more networks. This allows the cloud computing environment 1000 to offer software services to the one or more computer services to the one or more user computer systems 1002 and as such, a user computer system 1002 does not need to maintain resources locally.

In one embodiment, a node 1004 includes computer readable program instructions for carrying out various steps of various methods disclosed herein. In these embodiments, a user of a user computer system 1002 that is connected to the cloud computing environment may cause a node 1004 to execute the computer readable program instructions to carry out various steps of various methods disclosed herein.

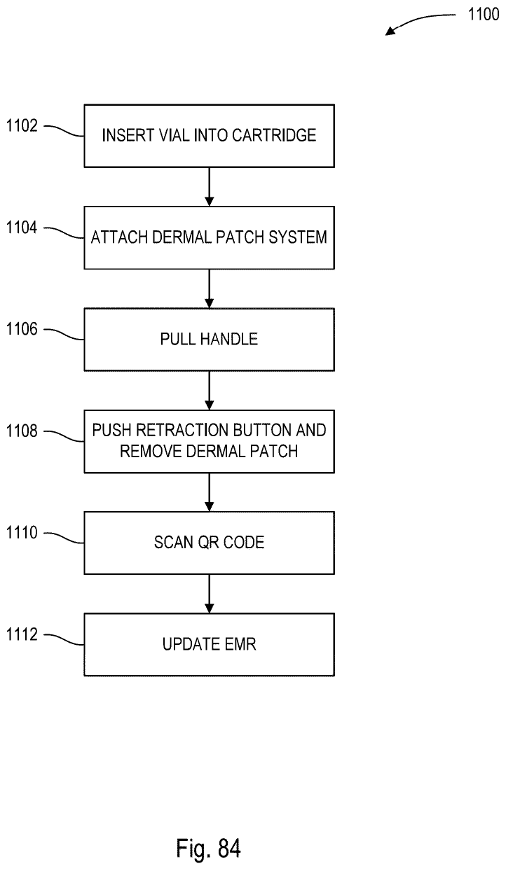

Referring now to FIG. 84, a method 1100 for administering a pharmaceutical is shown in accordance with an exemplary embodiment.

At 1102, as user inserts the vial 14 into the cartridge 12 as previously discussed herein.

At 1104, the user attaches the dermal patch system 10 to the skin of the subject via an adhesive film on a bottom surface of the dermal patch system 10 as previously discussed herein.

At 1106, the user of the dermal patch system 10 pulls the handle 302 of the pull mechanism 300 to deliver a pharmaceutical stored in the vial 14 to the subject as previously discussed herein. In some embodiments, the user can pull the handle 302 after placing the dermal patch system 10 in a proper orientation (e.g., by moving an arm of the subject to which the dermal patch system 10 is attached) as previously discussed herein.

At 1108, the user of the dermal patch system 10 pushes the retraction button 600 to remove the microneedles 536 from the skin of the subject and removes the dermal patch from the subject as previously discussed herein.

At 1110, the user of the dermal patch system 10 uses the computer system 32 to scan a QR code 30 of the dermal patch system 10 and associate the QR code 30 with an EMR 34 as previously discussed herein.

At 1112, the computer system 32 updates the EMR 34 to indicate that the subject was administered the pharmaceutical automatically or based on a user input as previously discussed herein.

As previously discussed, the above may be implemented by way of computer readable instructions, encoded or embedded on computer readable storage medium (which excludes transitory medium), which, when executed by a processor(s), cause the processor(s) to carry out the methods of the present disclosure.

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

What is claimed is:

1. A method for administering a pharmaceutical to a subject, the method comprising:
inserting a vial with the pharmaceutical into a cartridge,
attaching the cartridge to the subject's skin,
wherein the cartridge includes a pump and a plurality of microneedles, and pulling the pull mechanism,
wherein pulling the pull mechanism cause the microneedles to move from an undeployed position to a deployed position,
wherein the microneedles puncture the skin of the subject when in the deployed position, and
wherein pulling the pull mechanism causes the pump to pump the pharmaceutical to the microneedles thereby delivering the pharmaceutical to the subject.

2. The method of claim 1, wherein the pharmaceutical is a vaccine.

3. The method of claim 2, wherein a fractional dose of the vaccine is delivered to the subject.

4. The method of claim 1, wherein the pump primes the microneedles with an amount of pharmaceutical before the microneedles are moved to the deployed position.

5. The method of claim 1, wherein the cartridge further includes a tube that carries the pharmaceutical from the vial to the microneedles.

6. The method of claim 1, wherein the pump pumps the pharmaceutical from the vial and to the microneedles via positive displacement.
